(19) United States
(12) Patent Application Publication
(10) Pub. No.: US 2022/0168261 A1
(43) Pub. Date: Jun. 2, 2022

(54) COMPOUNDS AND METHODS FOR MANAGING CANCER THROUGH IMMUNE SYSTEM

(71) Applicant: AKAMARA THERAPEUTICS, INC., Philadelphia, PA (US)

(72) Inventors: Heena AGGARWAL, Delhi (IN); Shweta CHAUBEY, Delhi (IN); Rajat DESIKAN, Delhi (IN); Nimish GUPTA, Delhi (IN); Sonia JAIN, Delhi (IN); Seema MITTAL, Delhi (IN); Shiladitya SENGUPTA, Delhi (IN)

(21) Appl. No.: 17/430,849

(22) PCT Filed: Feb. 14, 2020
(86) PCT No.: PCT/US2020/018421

§ 371 Date: Aug. 13, 2021

(30) Foreign Application Priority Data
Feb. 14, 2019 (IN) 201911005924

(51) Int. Cl. A61K 31/282; A61P 35/00

(52) U.S. Cl. CPC A61K 31/282; A61P 35/00

(57) ABSTRACT

The present disclosure relates to methods of managing cancer by modulating/inhibiting cap methyltransferase enzyme. The modulation/inhibition is carried out by pharmacological or biological inhibitors, including but not limited to compounds of formula (VIII) or Compound A or A-1. Thus, the present disclosure relates to such inhibitors including compounds of formula (VIII) and their use for management of cancer. In some aspects, the compound of formula (VIII) is Compound 1. The present disclosure also relates to methods of activating B cells or T cells via modulation of cap methyltransferases. The present disclosure also relates to methods of managing cancer with a molecule or biologic that generates unmethylated RNA. The generation of unmethylated RNA activates B cells and presents the RNA to B-cell receptor (BCR). The present disclosure therefore also relates to management of cancer by activating B cells and then adoptively transferring the B cells to exert an anticancer effect.

### CROSS-REFERENCE TO RELATED APPLICATIONS

This application claims the benefit of Indian Provisional Patent Application No. 201911005924, filed Feb. 14, 2019, the disclosure of which is incorporated by reference herein.

### TECHNICAL FIELD

The present disclosure relates to methods of managing cancer by modulating/inhibiting cap methyltransferase enzyme. The modulation/inhibition is carried out by pharmacological or biological inhibitors, including but not limited to compounds described herein, such as compounds of formula (VIII) or compounds of formula A or A-1, or pharmaceutically acceptable salts thereof. Thus, the present disclosure relates to such inhibitors including compounds of formula (VIII) or compounds of formula A or A-1, or pharmaceutically acceptable salts thereof, and their use for management of cancer. In some embodiments, the present disclosure also relates to methods of activating B cells via modulation of cap methyltransferases. In other embodiments, the present disclosure also relates to methods of activating T cells via modulation of cap methyltransferases. The present disclosure also relates to methods of managing cancer with a molecule or biologic that generates unmethylated RNA. The generation of unmethylated RNA activates B cells and presents the RNA to B-cell receptor (BCR). The present disclosure therefore also relates to management of cancer by activating B cells and then adoptively transferring the B cells to exert an anticancer effect.

### BACKGROUND

Immuno-Oncology (I-O) is an innovative area of research that seeks to help the body's own immune system to fight cancer. Most of the work in this field revolves around T cell and its mechanism against cancer. However, in adaptive immunity, both T cells and B cells play an important role.

Prior studies have shown that pharmacological depletion of B cells increases the incidence of solid cancers. For example, some humanized monoclonal antibodies selectively deplete CD20-expressing B cells, causing neoplasms in high percentage of patients using such antibodies. This is a direct result of cancers caused by B cell depletion.

There is also evidence to show that several cancers including lymphoma, stomach, breast, bladder, and cervical epithelial cancer develop in patients with common variable immune deficiency (CVID) which is associated with the presence of defective humoral immunity. On the other hand, other studies provide pathological evidence that increase in intratumoral B cells increase disease free survival of the affected patients.

Thus, there is a need to arrive at therapies, treatments and/or inhibitors that not only focus on T cell, but also involve B cell and the related mechanisms and pathways for more efficient management of cancer.

### BRIEF DESCRIPTION OF THE FIGURES

In order that the disclosure may be readily understood and put into practical effect, reference will now be made to exemplary embodiments as illustrated with reference to the accompanying figure. The figure together with detailed description below, are incorporated in and form part of the specification, and serve to further illustrate the embodiments and explain various principles and advantages, in accordance with the present disclosure where:

FIG. 1 depicts the proteomics workflow for elucidation of binding of Compound 1 to CMTR2.

FIG. 2 depicts binding of CMTR2 to Compound 1 by ELISA.

FIG. 3 depicts the effect of blocking of CMTR2 function on CMTR2 levels.

FIG. 4 depicts iBlot assays with IFIT1 and presence of IFIT1-mRNA complex.

FIG. 5 depicts the differential mRNA expression profile of the interferon pathway in 4T1 breast cancer.

FIG. 6 depicts fold change in transcript levels of B cells after 24 h co-culture with Compound 1 treated 4T1 cells than with non-treated 4T1 cells. Each grouping of 4 bars follows the order, from left to right, (i) control, (ii) 10 μM of Compound 1, (iii) 20 μM of Compound 1, and (iv) 30 μM of Compound 1.

FIG. 7 depicts fold change in transcript levels of B cells after 24 h co-culture with Compound 1 treated 4T1 cells than with non-treated 4T1 cells. Each grouping of 4 bars follows the order, from left to right, (i) control, (ii) 10 μM of Compound 1, (iii) 20 μM of Compound 1, and (iv) 30 μM of Compound 1.

FIGS. 8A-8E depict effect of Compound 1 on tumor regression in established 4T1 tumors in immunocompetent syngeneic BALB/c mice. Specifically, FIG. 8A is a line graph showing tumor size as a function of time. FIG. 8B are images of tumor splices (days 3, 4, and 5) for mice treated with control or compound 1. FIGS. 8C to 8E are bar graphs showing the RTPCR analysis of transcripts of biomarkers.

FIG. 9 depicts the gene expression in 4T1 tumors and effect of Compound 1 treatment.

FIG. 10 depicts the dose-dependent effect on tumor post treatment with Compound 1.

FIG. 11 depicts the effect on tumor growth post rechallenge with cancer cells.

FIG. 12 depicts the changes in B cell subsets in lymph node and spleen of tumor regressed mice

FIG. 13 depicts the effect on tumor progression in the B cell adoptive transfer groups.

FIG. 14 depicts the effect of different dose regimens of Compound 1 on tumor growth in a immunocompetent Lewis lung carcinoma model.

FIGS. 15A and 15B depict self-assembled liposomal membrane, Cryo-EM images of the supramolecular liposomes and FIG. 15C depicts the distribution of size as measured by dynamic laser light scattering.

FIG. 16 is bar graph showing the effect of concentration of Compound 1 on the relative mRNA expression (% control) for INF-α, IRF7, TRIF, and TLR3.

FIG. 17 is a dot plot showing the effect of Compound 1 (varying concentrations) on the CTCF.

FIG. 18A is an iBlot assay in the absence and presence of Compound 1 showing intracellular INF-α and GAPDH levels. FIG. 18B is a bar graph showing the effect of Compound 1 on the intensity normalized to the loading control.

FIG. 19A is an iBlot assay in the absence and presence of Compound 1 showing extracellular INF-α and Ponceau-S levels. FIG. 19B is a bar graph showing the effect of Compound 1 on the intensity normalized to the loading control.

FIG. 20 are iBlot (WB) assays showing the effect of isolated IFIT1-mRNA complex from untreated and Compound 1 treated 4T1 cells on B-cells activation.

FIG. 21 is a line graph showing the effect of Compound 1 on change in body weight (%) up to 29 days post-implantation.

FIG. 22 is a bar graph showing the IFIT1 levels in plasma on days 3, 4, 5, and 8 as measured by dot-blot.

FIG. 23 is a western blot analysis of oligo-dT pull downs from plasma showing binding of circulating IFIT1 to mRNA.

FIG. 24 is a bar graph showing the INF-α levels in plasma on days 3, 4, 5, and 8 as measured by dot-blot.

FIG. 25 is a dot plot showing % B-cell on day 3 after treatment with Compound 1.

FIG. 26 is a line graph showing the effect of Compound 1 on tumor volume (mm³) in T and B-cell knockout (SCID) mice.

FIG. 27 are line graphs showing the effect of Compound 1 on tumor volume (mm³) after re-implantation of cancer cells into mice. in immunocompetent (wild-type), B-cell knockout (JH), and T and B-cell knockout (SCID) mice.

FIG. 28 is a line graph showing the effect tumor volume (mm³) after injection of B-cells harvested from mice in which tumor regressed post Compound 1 treatment.

### DETAILED DESCRIPTION

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The use of the expression “at least” or “at least one” suggests the use of one or more elements or ingredients or quantities, as the use may be in the embodiment of the disclosure to achieve one or more of the desired objects or results. Throughout this specification, the word “comprise”, or variations such as “comprises” or “comprising” or “containing” or “has” or “having” wherever used, will be understood to imply the inclusion of a stated element, integer or step, or group of elements, integers or steps, but not the exclusion of any other element, integer or step, or group of elements, integers or steps.

It is also to be understood that the phrases or terms employed herein are for the purpose of description and not intended to be of any limitation. Throughout the present disclosure, the word “comprise”, or variations such as “comprises” or “comprising” wherever used, are to be understood to imply the inclusion of a stated element, integer or step, or group of elements, integers or steps, but not the exclusion of any other element, integer or step, or group of elements, integers or steps.

Where a numerical limit or range is stated herein, the endpoints are included. Also, values and sub-ranges within a numerical limit or range are specifically included as if explicitly written out.

With respect to the use of any plural and/or singular terms in the present disclosure, those of skill in the art can translate from the plural to the singular and/or from the singular to the plural as is considered appropriate to the context and/or application. The various singular/plural permutations may be expressly set forth herein for the sake of clarity.

As used herein, the term “managing” or “management” includes, treating or healing of a disease condition or disorder or ill effects or side effects. The term also encompasses maintenance of the optimum state and prevention of the further progress in the disease condition or disorder or ill effects or side effects. Further, “management” or “managing” refers to decreasing the risk of death due to a disease or disorder, delaying the onset of a disease or disorder, inhibiting the progression of a disease or disorder, partial or complete cure of a disease or disorder and/or adverse effect attributable to the disease or disorder, obtaining a desired pharmacologic and/or physiologic effect (the effect may be prophylactic in terms of completely or partially preventing a disorder or disease or condition, or a symptom thereof and/or may be therapeutic in terms of a partial or complete cure for a disease or disorder and/or adverse effect attributable to the disease or disorder), or relieving a disease or disorder (i.e. causing regression of the disease or disorder). In some cases, the terms “managing” or “management” are interchangeably used with terms “treating” or “treatment”, and are intended to convey the ordinary meaning as understood by a person skilled in the art.

The term “alkyl” as used herein refers to straight- and branched-chain saturated aliphatic hydrocarbon groups. In some embodiments, an alkyl group has 1 to about 12 carbon atoms. In other embodiments, an alkyl group has 1 to about 6 carbon atoms. In further embodiments, an alkyl group has 1 to about 4 carbon atoms.

The term “hydroxyl” or “hydroxy” as used herein refers to —OH.

The term “alkoxy” as used herein refers to the O-(alkyl) group, where the point of attachment is through the oxygen-atom and the alkyl group is defined above.

The term “thiol” as used herein refers to —SH.

The term “thioalkyl” as used herein refers to the S-(alkyl) group, where the point of attachment is through the sulfur-atom and the alkyl group is defined above.

The term “amino” as used herein refers to —NH₂. The term “alkylamino” as used herein refers to a NH(alkyl) group where the point of attachment is through the nitrogen-atom and the alkyl group is defined above. Similarly, the term “dialkylamino” refers to a NH(alkyl)(alkyl) group, where the alkyl groups can be the same or different, the point of attachment is through the nitrogen-atom and the alkyl group is defined above.

The term “halogen” as used herein refers to Cl, Br, F, or I groups. In some embodiments, halogen refers to Cl. In some embodiments, halogen refers to Br. In some embodiments, halogen refers to F. In some embodiments, halogen refers to I.

The term “cyclyl” as used herein refers to a cyclic aliphatic having 3 to 8 carbon atoms, e.g., 3, 4, 5, 6, 7, or 8 carbon atoms and includes, for example, cyclopropyl, cyclobutyl, cyclopentyl, cyclohexyl, cycloheptyl, or cyclooctyl. The cyclyl may be unsubstituted or substituted.

“Aryl” as used herein refers to 6-15 membered monoradical bicyclic or tricyclic hydrocarbon ring systems, including bridged, spiro, and/or fused ring systems, in which at least one of the rings is aromatic. An aryl group may contain 6 (i.e., phenyl), about 9 to about 15 ring atoms, or about 9 to about 11 ring atoms. In some embodiments, aryl includes, but is not limited to, phenyl, naphthyl, indanyl, indenyl, anthryl, phenanthryl, fluorenyl, 1,2,3,4-tetrahydronaphthalenyl, 6,7,8,9-tetrahydro-5H-benzocycloheptenyl, and 6,7,8,9-tetrahydro-5H-benzocycloheptenyl.

“Heterocyclyl” as used herein refers to a stable 3- to 18-membered non-aromatic ring radical that comprises 2 to about 12 carbon atoms and from 1 to 6 heteroatoms selected from nitrogen, oxygen and sulfur. The heterocyclyl radical is a monocyclic, bicyclic, tricyclic or tetracyclic ring system, which may include fused or bridged ring systems. The heteroatoms in the heterocyclyl radical may be optionally oxidized. One or more nitrogen atoms, if present, are optionally quaternized. The heterocyclyl radical is partially or fully saturated. The heterocyclyl may be attached to the rest of the molecule through any atom of the ring(s). Examples of heterocyclyl radicals include, but are not limited to, azepanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. “Heterocyclyl” also includes bicyclic ring systems wherein one non-aromatic ring, usually with 3 to 7 ring atoms, contains at least 2 carbon atoms in addition to 1-3 oxygen, sulfur, or nitrogen, as well as combinations comprising at least one of the foregoing heteroatoms; and the other ring, usually with 3 to 7 ring atoms, optionally contains 1-3 oxygen, sulfur, or nitrogen and is not aromatic.

“Heteroaryl” as used herein refers to a 5- to 18-membered aromatic radical that includes one or more ring heteroatoms selected from nitrogen, oxygen and sulfur, and which may be a monocyclic, bicyclic, tricyclic or tetracyclic ring system. A polycyclic heteroaryl group may be fused or non-fused. The heteroatom(s) in the heteroaryl radical are optionally oxidized. One or more nitrogen atoms, if present, are optionally quaternized. The heteroaryl may be attached to the rest of the molecule through any atom of the ring(s). Examples of heteroaryls include, but are not limited to, azepinyl, acridinyl, benzimidazolyl, benzindolyl, 1,3-benzodioxolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzodioxolyl, benzodioxinyl, benzoxazolyl, benzopyranyl, benzopyranonyl, benzofuranyl, benzofuranonyl, benzofurazanyl, benzothiazolyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl).

The term “acyl” as used herein refers to a C(O)(alkyl) or C(O)(O-alkyl) group, where the point of attachment is through the carbon atom and the alkyl group is defined above.

In some embodiments, the present disclosure relates to methods of inhibiting cap methyltransferase enzyme 2 using the compounds of formula (VIII), such as Compound 1, or pharmaceutically acceptable salts thereof.

In further embodiments, the present disclosure relates to methods of inhibiting cap methyltransferase enzyme 2 using compounds A or A-1, or pharmaceutically acceptable salts thereof.

In other embodiments, the present disclosure relates to methods of managing cancer by modulating/inhibiting cap methyltransferase enzyme. The modulation/inhibition is carried out by pharmacological or biological inhibitors, including but not limited to compound of formula (VIII) or Compound A or A-1, or pharmaceutically acceptable salts thereof. Thus, the present disclosure relates to such inhibitors including compound of formula (VIII), or pharmaceutically acceptable salts thereof, and their use for managing or treating cancer. The present disclosure further relates to Compound A or A-1, or pharmaceutically acceptable salts thereof, and their use for managing or treating cancer.

In yet other embodiments, the present disclosure provides methods of activating B cells via modulating cap methyltransferases using the compound of formula (VIII), such as Compound 1, or pharmaceutically acceptable salts thereof.

In still further embodiments, the present disclosure provides methods of activating B cells via modulating cap methyltransferases using Compound A or A-1, or pharmaceutically acceptable salts thereof.

In further embodiments, the present disclosure provides methods of activating T cells via modulating cap methyltransferases using the compound of formula (VIII), such as Compound 1, or pharmaceutically acceptable salts thereof.

In other embodiments, the present disclosure provides methods of activating T cells via modulating cap methyltransferases using Compound A or A-1, or pharmaceutically acceptable salts thereof.

In still other embodiments, the present disclosure provides methods of managing, treating, or regressing cancer with a compound of formula (VIII), such as Compound 1, or pharmaceutically acceptable salts thereof, that generates unmethylated RNA.

In further embodiments, the present disclosure provides methods of managing, treating, or regressing cancer with Compound A or A-1, or pharmaceutically acceptable salts thereof, that generates unmethylated RNA.

In yet further embodiments, the present disclosure provides methods for activating B cells using the compound of formula (VIII), such as Compound 1, or pharmaceutically acceptable salts thereof, and then adoptively transferring the B cells to exert an anticancer effect.

In still other embodiments, the present disclosure provides methods for activating B cells using Compound A or A-1, or pharmaceutically acceptable salts thereof, and then adoptively transferring the B cells to exert an anticancer effect.

In other embodiments, the present disclosure provides methods for managing or treating cancer by upregulating B cell mediated immune response caused by B cells activation and differentiation upregulated using the compounds of formula (VIII), such as Compound 1, or pharmaceutically acceptable salts thereof.

In still further embodiments, the present disclosure provides methods for managing or treating cancer by upregulating B cell mediated immune response caused by B cells activation and differentiation upregulated using Compounds A or A-1, or pharmaceutically acceptable salts thereof.

In further embodiments, the present disclosure relates to methods of treating cancer by activating the B cell mediated immune response, orchestrated by interaction of the compounds of formula (VIIII), such as Compound 1, or pharmaceutically acceptable salts thereof, with nucleic acids.

In other embodiments, the present disclosure relates to methods of treating cancer by activating the B cell mediated immune response, orchestrated by interaction of Compound A or A-1, or pharmaceutically acceptable salts thereof, with nucleic acids.

In still further embodiments, the present disclosure provides methods for inducing an immune response using the compounds of formula (VIII), such as Compound 1, or pharmaceutically acceptable salts thereof.

In other embodiments, the present disclosure provides methods for inducing an immune response using the compound (A) or A-1 or pharmaceutically acceptable salts thereof.

In yet other embodiments, the present disclosure provides methods for inducing immune memory using the compounds of formula (VIII), or pharmaceutically acceptable salts thereof.

In still other embodiments, the present disclosure provides methods for inducing immune memory using the compound of formula (VIII), such as Compound 1, or pharmaceutically acceptable salts thereof.

In yet other embodiments, the present disclosure provides methods for inducing immune memory using Compound A or A-1, or pharmaceutically acceptable salts thereof.

As used herein, a compound of formula (VIII) refers to Q-linker-lipid (VIII), wherein Q is a platinum containing moiety and the linker has at least one linkage to the platinum atom, such as Compound 1.

In some aspects, Q is

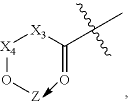

wherein, X₃ is (CH₂)ₙ, CH₂—NH, or C₄H₈; X₄ is CO or —CH(CH₃)—; Z is a platinum containing compound, wherein the platinum forms a part of the ring; and n is 0, 1, or 2. In some embodiments, X₃ is (CH₂)ₙ. In other embodiments, X₃ is CH₂—NH. In further embodiments, X₃ is C₄H₈. In yet other embodiments, X₄ is CO. In still further embodiments, X₄ is —CH(CH₃)—. In other embodiments, n is 0. In further embodiments, n is 1. In still other embodiments, n is 2.

In other aspects, Q is

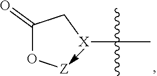

wherein, X is NH or N(CH₂COO⁻); and Z is a platinum containing compound, wherein the platinum forms a part of the ring. In some embodiments, X is NH. In other embodiments, X is N(CH₂COO⁻).

In further aspects, Q is

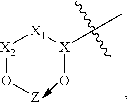

wherein, X is S⁺, C, S⁺═O, N⁺H, or P═O; X₁ is —CH—, —CH₂— or —CH₂O—; X₂ is C(O); and Z is a platinum containing compound, wherein the platinum forms a part of the ring. In some embodiments, X is S⁺. In other embodiments, X is C. In further embodiments, X is S⁺═O. In still other embodiments, X is N⁺H. In yet further embodiments, X is P═O. In some embodiments, X₁ is —CH—. In other embodiments, X₁ is —CH₂—. In further embodiments, X₁ is —CH₂—.

In yet other aspects, Q is

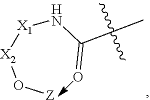

wherein, X₁ is —(CH₂)ₙ—; X₂ is C(O); Z is a platinum containing compound, wherein the platinum forms a part of the ring; and n is 0, 1, or 2. In some embodiments, n is 0. In other embodiments, n is 1. In further embodiments, n is 2.

In some embodiments, the platinum is coordinated to a leaving group via a unique O—Pt monocarboxylato covalent bond and a ═O→Pt coordinate bond. Further, the present disclosure also discloses platinum based compounds wherein the platinum is coordinated to a leaving group via O—Pt monocarboxylato or dicarboxylato covalent bond(s). In other embodiments, the platinum moiety is a platinum (II) or platinum (IV) compound. In further embodiments, the platinum (II) compound is DACH-platinum, cisplatin, oxaliplatin, carboplatin, paraplatin, satraplatin, or combinations thereof. In other embodiments, the platinum containing compound is a Pt(II) compound, Pt(IV) compound or halide containing platinum compound. In a preferred embodiment, the platinum compound is oxaliplatin.

In some aspects, Z is

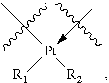

wherein, R₁ and R₂ are, independently, halogen, alkyl, amino, alkylamino, dialkylamino, hydroxyl, alkoxy, thiol, thioalkyl, O-acyl, or combinations thereof. In some embodiments, R₁ and R₂, together with the Pt atom form an optionally substituted cyclyl or heterocyclyl.

In other aspects, Z is

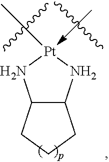

wherein, p is 0, 1, 2, or 3. In some embodiments, p is 0. In other embodiments, p is 1. In further embodiments, p is 2. In yet other embodiments, p is 3.

In other aspects, Z is

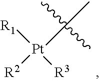

wherein, R¹, R² and R³ are, independently, halogen, alkyl, amino, alkylamino, dialkylamino, hydroxyl, alkoxy, thiol, thioalkyl, O-acyl, linker-lipid, or combinations thereof. In some embodiments, R₁ and R₂ together with the Pt atom or R₂ and R₃ together with the Pt atom form an optionally substituted cyclyl or heterocyclyl. In other embodiments, R₁ and R₂ together with the Pt atom and R₂ and R₃ together with the Pt atom form an optionally substituted cyclyl or heterocyclyl.

In further aspects, Z is

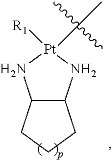

wherein, R₁ is halogen, alkyl, amino, alkylamino, dialkylamino, hydroxyl, alkoxy, thiol, thioalkyl, O-acyl, or combinations thereof, and p is 0, 1, 2, or 3. In some embodiments, R₁ is halogen —Cl, —NCS, —O═S(CH₃)₂, —SCH₃, or -linker-lipid. In other embodiments, p is 2.

In yet other aspects, Z is

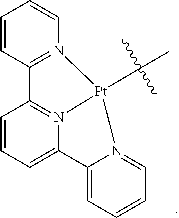

In still further aspects, Z is

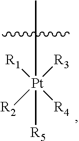

wherein, R₁, R₂, R₃, R₄ and R₅ are, independently, halogen, alkyl, amino, alkylamino, dialkylamino, hydroxyl, alkoxy, thiol, thioalkyl, O-acyl, -linker-lipid, or combinations thereof. In some embodiments, R₁ and R₂ together with the Pt atom form an optionally substituted cyclyl or heterocyclyl. In other embodiments, R₁ and R₂ together with the Pt atom form an optionally substituted cyclyl or heterocyclyl. In yet further embodiments, R₁ and R₂ together with the Pt atom form an optionally substituted cyclyl or heterocyclyl and R₃ and R₄ together with the Pt atom form an optionally substituted cyclyl or heterocyclyl. In still other embodiments, R₅ is OH, OC(O)CH₃, or OC(O)-phenyl.

In other aspects, Z is

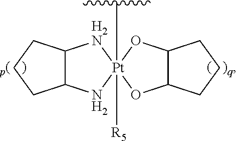

wherein, p and q are, independently, 0, 1, 2, or 3; and R₅ is OH, OC(O)CH₃, or OC(O)-phenyl. In some embodiments, p is 2. In other embodiments, q is 2. In further embodiments, p and q are both 2.

In further aspects, Z is

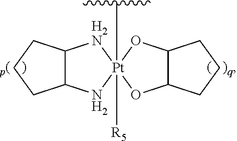

wherein, p and q are both 2; and R₅ is OH, OC(O)CH₃, or OC(O)-phenyl.

In some embodiments, the platinum (II) compound comprises at least two nitrogen atoms, where said nitrogen atoms are directly linked to platinum. In a further embodiment, the two nitrogen atoms are linked to each other via an optionally substituted linker, e.g. acyclic or cyclic linker. The term “cyclic linker” refers to a linking moiety that comprises at least one ring structure. Examples of cyclic linkers include, without limitation, aryl, heteroaryl, cyclyl or heterocyclyl.

In some aspects, Q is

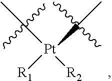

wherein, R₁ and R₂ are, independently, halogen, alkyl, amino, alkylamino, dialkylamino, hydroxyl, alkoxy, thiol, thioalkyl, O-acyl, or any combinations thereof. In some embodiments, R₁ and R₂, together with the Pt atom form an optionally substituted cyclyl or heterocyclyl.

In other aspects, Q is

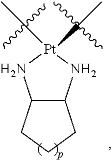

wherein, p is 0, 1, 2, or 3. In some embodiments, p is 2.

In further aspects, Q is

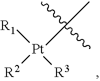

wherein, R¹, R² and R³ are, independently, halogen, alkyl, amino, alkylamino, dialkylamino, hydroxyl, alkoxy, thiol, thioalkyl, O-acyl, or combinations thereof. In some embodiments, R₁ and R₂ together with the Pt atom or R₂ and R₃ together with the Pt atom form an optionally substituted cyclyl or heterocyclyl. In other embodiments, R₁ and R₂ together with the Pt atom and R₂ and R₃ together with the Pt atom form an optionally substituted cyclyl or heterocyclyl.

In yet other aspects, Q is

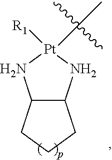

wherein, R₁ is halogen, alkyl, amino, alkylamino, dialkylamino, hydroxyl, alkoxy, thiol, thioalkyl, O-acyl, or combinations thereof, and p is 0, 1, 2, or 3. In some embodiments, R₁ is halogen —Cl, —NCS, —O═S(CH₃)₂, —SCH₃, or -linker-lipid. In other embodiments, p is 2.

In still further aspects, Q is

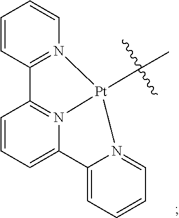

In other aspects, Q is

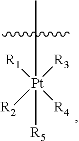

wherein, R₁, R₂, R₃, R₄ and R₅ are, independently, halogen, alkyl, amino, alkylamino, dialkylamino, hydroxyl, alkoxy, thiol, thioalkyl, O-acyl, or combinations thereof. In some embodiments, R₁ and R₂ together with the Pt atom form an optionally substituted cyclyl or heterocyclyl. In other embodiments, R₁ and R₂ together with the Pt atom form an optionally substituted cyclyl or heterocyclyl. In further embodiments, R₁ and R₂ together with the Pt atom form an optionally substituted cyclyl or heterocyclyl and R₃ and R₄ together with the Pt atom form an optionally substituted cyclyl or heterocyclyl. In still other embodiments, R₅ is OH, OC(O)CH₃, or OC(O)-phenyl.

In further aspects, Q is

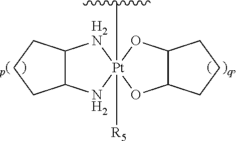

Wherein, p and q are, independently, 0, 1, 2, or 3; and R₅ is OH, OC(O)CH₃, or OC(O)-phenyl. In some embodiments, p is 2. In other embodiments, q is 2. In further embodiments, p and q are both 2.

In further aspects, Q is

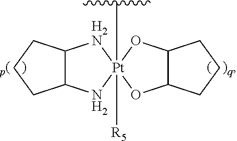

wherein, p and q are both 2; and R₅ is OH, OC(O)CH₃, or OC(O)-phenyl.

The term “lipid” as used herein is used in the conventional sense and includes compounds of varying chain length, from as short as about 2 carbon atoms to as long as about 28 carbon atoms. The compounds are saturated or unsaturated, in the form of straight- or branched-chains, or in the form of unfused or fused ring structures. Exemplary lipids include, but are not limited to, a fat, wax, sterol, steroid, bile acid, fat-soluble vitamin (such as A, D, E, and K), monoglyceride, diglyceride, phospholipid, glycolipid, sulpholipid, aminolipid, chromolipid (lipochrome), glycerophospholipid, sphingolipid, prenollipid, saccharolipid, polyketide, or fatty acid. In some embodiments, the lipid is a sterol lipid, fatty acid, fatty alcohol, glycerolipid (e.g., monoglyceride, diglyceride, or triglyceride), phospholipid, glycerophospholipid, sphingolipid, prenol lipid, saccharolipid, polyketide, or any combination thereof. In other embodiments, the lipid is a polyunsaturated fatty acid or alcohol. The term “polyunsaturated fatty acid” or “polyunsaturated fatty alcohol” as used herein means a fatty acid or alcohol with two or more carbon-carbon double bonds in its hydrocarbon chain. In further embodiments, the lipid is a highly unsaturated fatty acid or alcohol. The term “highly polyunsaturated fatty acid” or “highly polyunsaturated fatty alcohol” as used herein refers to a fatty acid or alcohol having at least 18 carbon atoms and at least 3 double bonds. In yet other embodiments, the lipid is an omega-3 fatty acid. The term “omega-3 fatty acid” as used herein refers to a polyunsaturated fatty acid, where the first double bond occurs at the third carbon-carbon bond from the end opposite the acid group.

In some preferred embodiments, the lipid is 1,3-propanediol dicaprylate/dicaprate; 10-undecenoic acid; 1-dotriacontanol; 1-heptacosanol; 1-nonacosanol; 2-ethyl hexanol; an androstane; arachidic acid; arachidonic acid; arachidyl alcohol; behenic acid; behenyl alcohol; Capmul MCM C₁₀; capric acid; capric alcohol; capryl alcohol; caprylic acid; caprylic/capric acid ester of saturated fatty alcohol C₁₂-C₁₈; caprylic/capric triglyceride; caprylic/capric triglyceride; ceramide phosphorylcholine (Sphingomyelin, SPH); ceramide phosphorylethanolamine (Sphingomyelin, Cer-PE); ceramide phosphorylglycerol; ceroplastic acid; cerotic acid; cerotic acid; ceryl alcohol; cetearyl alcohol; Ceteth-10; cetyl alcohol; a cholane; a cholestane; cholesterol; cis-11-eicosenoic acid; cis-11-octadecenoic acid; cis-13-docosenoic acid; cluytyl alcohol; coenzyme Q10 (CoQ10); dihomo-γ-linolenic; docosahexaenoic acid; egg lecithin; eicosapentaenoic acid; eicosenoic acid; elaidic acid; elaidolinolenyl alcohol; elaidolinoleyl alcohol; elaidyl alcohol; erucic acid; erucyl alcohol; estranes; ethylene glycol distearate (EGDS); geddic acid; geddyl alcohol; glycerol distearate (type I) EP (Precirol ATO 5); glycerol tricaprylate/caprate; glycerol tricaprylate/caprate (CAPTEX©355 EP/NF); glyceryl monocaprylate (Capmul MCM C8 EP); glyceryl triacetate; glyceryl tricaprylate; glyceryl tricaprylate/caprate/laurate; glyceryl tricaprylate/tricaprate; glyceryl tripalmitate (Tripalmitin); henatriacontylic acid; heneicosyl alcohol; heneicosylic acid; heptacosylic acid; heptadecanoic acid; heptadecyl alcohol; hexatriacontylic acid; isostearic acid; isostearyl alcohol; lacceroic acid; lauric acid; lauryl alcohol; lignoceric acid; lignoceryl alcohol; linoelaidic acid; linoleic acid; linolenyl alcohol; linoleyl alcohol; margaric acid; mead; melissic acid; melissyl alcohol; montanic acid; montanyl alcohol; myricyl alcohol; myristic acid; myristoleic acid; myristyl alcohol; neodecanoic acid; neoheptanoic acid; neononanoic acid; nervonic; nonacosylic acid; nonadecyl alcohol; nonadecylic acid; nonadecylic acid; oleic acid; oleyl alcohol; palmitic acid; palmitoleic acid; palmitoleyl alcohol; pelargonic acid; pelargonic alcohol; pentacosylic acid; pentadecyl alcohol; pentadecylic acid; phosphatidic acid (phosphatidate, PA); phosphatidylcholine (lecithin, PC); L-α-phosphatidylcholine, hydrogenated (soy); phosphatidylethanolamine (cephalin, PE); phosphatidylinositol (PI); phosphatidylinositol bisphosphate (PIP2); phosphatidylinositol phosphate (PIP); phosphatidylinositol triphosphate (PIP3); phosphatidylserine (PS); polyglyceryl-6-distearate; a pregnane; propylene glycol dicaprate; propylene glycol dicaprylocaprate; propylene glycol dicaprylocaprate; psyllic acid; recinoleaic acid; recinoleyl alcohol; sapienic acid; soy lecithin; stearic acid; stearidonic; stearyl alcohol; tricosylic acid; tridecyl alcohol; tridecylic acid; triolein; undecyl alcohol; undecylenic acid; undecylic acid; vaccenic acid; α-linolenic acid; γ-linolenic acid; a fatty acid salt of 10-undecenoic acid, adapalene, arachidic acid, arachidonic acid, behenic acid, butyric acid, capric acid, caprylic acid, cerotic acid, cis-11-eicosenoic acid, cis-11-octadecenoic acid, cis-13-docosenoic acid, docosahexaenoic acid, eicosapentaenoic acid, elaidic acid, erucic acid, heneicosylic acid, heptacosylic acid, heptadecanoic acid, isostearic acid, lauric acid, lignoceric acid, linoelaidic acid, linoleic acid, montanic acid, myristic acid, myristoleic acid, neodecanoic acid, neoheptanoic acid, neononanoic acid, nonadecylic acid, oleic acid, palmitic acid, palmitoleic acid, pelargonic acid, pentacosylic acid, pentadecylic acid, recinoleaic acid (e.g. zinc recinoleate), sapienic acid, stearic acid, tricosylic acid, tridecylic acid, undecylenic acid, undecylic acid, vaccenic acid, valeric acid, α-linolenic acid, γ-linolenic acid; or any combination thereof. In some embodiments, the lipid is cholesterol or alpha tocopherol. In other embodiments, the lipid is cholesterol.

As used herein, the term “linker” means an organic moiety that connects two parts of a compound. Linkers typically comprise a direct bond or an atom such as oxygen or sulfur, a unit such as NR¹, C(O), C(O)NH, C(O)O, NHC(O)O, OC(O)O, SO, SO₂, SO₂NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO₂, NR¹, C(O), C(O)NH, C(O)O, NHC(O)O, OC(O)O, SO₂NH, cleavable linking group, substituted or unsubstituted aryl, substituted or unsubstituted heteroaryl, substituted or unsubstituted heterocyclic; where R¹ is hydrogen, acyl, aliphatic or substituted aliphatic.

In some embodiments, the linker is a branched linker. The branchpoint of the branched linker may be at least trivalent, but can be a tetravalent, pentavalent or hexavalent atom, or a group presenting such multiple valencies. In some embodiments, the branchpoint is —N, —N(Q)-C, —O—C, —S—C, —SS—C, —C(O)N(Q)-C, —OC(O)N(Q)-C, —N(Q)C(O)—C, or —N(Q)C(O)O—C; wherein Q is independently for each occurrence H or optionally substituted alkyl. In some embodiments, the branchpoint is glycerol or derivative thereof.

A cleavable linking group is one which is sufficiently stable outside the cell, but which upon entry into a target cell is cleaved to release the two parts the linker is holding together. In a preferred embodiment, the cleavable linking group is cleaved at least 10 times or more, preferably at least 100 times faster in the target cell or under a first reference condition (which can, e.g., be selected to mimic or represent intracellular conditions) than in the blood or serum of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; amidases; endosomes or agents that can create an acidic environment, e.g., those that result in a pH of five or lower; enzymes that can hydrolyze or degrade an acid cleavable linking group by acting as a general acid, peptidases (which can be substrate specific) and proteases, and phosphatases.

A linker can include a cleavable linking group that is cleavable by a particular enzyme. The type of cleavable linking group incorporated into a linker can depend on the cell to be targeted. For example, liver targeting ligands can be linked to the cationic lipids through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis. Linkers that contain peptide bonds can be used when targeting cell types rich in peptidases, such as liver cells and synoviocytes.

In some embodiments, cleavable linking group is cleaved at least 1.25, 1.5, 1.75, 2, 3, 4, 5, 10, 25, 50, or 100 times faster in the cell (or under in vitro conditions selected to mimic intracellular conditions) as compared to blood or serum (or under in vitro conditions selected to mimic extracellular conditions). In some embodiments, the cleavable linking group is cleaved by less than 90%, 80%, 70%, 60%, 50%, 40%, 30%, 20%, 10%, 5%, or 1% in the blood (or in vitro conditions selected to mimic extracellular conditions) as compared to in the cell (or under in vitro conditions selected to mimic intracellular conditions).

Exemplary cleavable linking groups include, but are not limited to, redox cleavable linking groups (e.g., —S—S— and —C(R)₂—S—S—, wherein R is H or C₁-C₆ alkyl and at least one R is C₁-C₆ alkyl such as CH₃ or CH₂CH₃); phosphate-based cleavable linking groups (e.g., —O—P(O)(OR)—O—, —O—P(S)(OR)—O—, —O—P(S)(SR)—O—, —S—P(O)(OR)—O—, —O—P(O)(OR)—S—, —S—P(O)(OR)—S—, —O—P(S)(ORk)-S—, —S—P(S)(OR)—O—, —O—P(O)(R)—O—, —O—P(S)(R)—O—, —S—P(O)(R)—O—, —S—P(S)(R)—O—, —S—P(O)(R)—S—, —O—P(S)(R)—S—, —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, and —O—P(S)(H)—S—, wherein R is optionally substituted linear or branched C₁-C₁₀ alkyl); acid cleavable linking groups (e.g., hydrazones, esters, and esters of amino acids, —C═NN— and —OC(O)—); ester-based cleavable linking groups (e.g., —C(O)O—); peptide-based cleavable linking groups, (e.g., linking groups that are cleaved by enzymes such as peptidases and proteases in cells, e.g., —NHCHR^{A}C(O)NHCHR^{B}C(O)—, where R^{A} and R^{B} are the R groups of the two adjacent amino acids). A peptide based cleavable linking group comprises two or more amino acids. In some embodiments, the peptide-based cleavage linkage comprises the amino acid sequence that is the substrate for a peptidase or a protease found in cells.

In some embodiments, an acid cleavable linking group is cleavable in an acidic environment with a pH of about 6.5 or lower (e.g., about 6.5, 6.0, 5.5, 5.0, or lower), or by agents such as enzymes that can act as a general acid.

Linkers according to the present invention include moieties comprising two or more carbon molecules such as, for example, ethylenediamine, ethyleneglycol, glycine, beta-alanine and polyethylene glycol (PEG) of molecular weight about 44 to about 200 kD. Further, it is to be understood from the present disclosure that the platinum moiety and/or the lipid may be modified to comprise functional groups for linking to the linker molecule.

In some embodiments, the linker is —X—CH₂—X₂—X₁—, wherein X is NH; X₁ is C(O)O, C(O)NH, O(CH₂)—O, NH, or O; X₂ is (CH₂)ₙ or C(O); and n is 0, 1, 2, 3, 4, or 5. In other embodiments, the linker is —(CH₂)ₙO—, —(CH₂)ₙNHC(O)O—, —(CH₂)ₙOC(O)NH—, —(CH₂)ₙC(O)NH(CH₂)ₘO—, —(CH₂)ₙO(CH₂)ₘO—, —(CH₂)ₙC(O)—, —(CH₂)ₙNHC(O)(CH₂)ₘO—, or —(CH₂)ₙC(O)O—; and n and m are independently 0, 1, 2, 3, 4, or 5. In further embodiments, the linker is X₃—X₄X₅—X₆—, wherein X₃ is CH, CH₂, or O; and X₄, X₅ and X₆ are independently same or different and are —CH₂O— or O. In yet other embodiments, the linker is —CH₂O—. In still further embodiments, the linker is a bond, —O—, NHCH₂CH₂NHC(O)—, —NHCH₂CH₂NHC(O)O, —NHCH₂CH₂, NHCH₂CH₂O—, —NHCH₂C(O)—, —NHCH₂C(O)O—, —NHCH₂C(O)OCH₂CH₂CH₂—, —NHCH₂C(O)OCH₂CH₂CH₂O—, —NHCH₂C(O)NH—, —CH₂CH₂, —CH₂CH₂O—, —CH₂CH₂NHC(O)—, —CH₂CH₂NHC(O)O—, —CH₂CH₂O—, —CH₂C(O)NHCH₂CH₂—, —CH₂C(O)NHCH₂CH₂O—, —CH₂CH₂OCH₂CH₂—, —CH₂CH₂OCH₂CH₂O—, —CH₂C(O)—, —CH₂C(O)O—, —CH₂CH₂CH₂—, —CH₂CH₂CH₂O—, ═CHCH═CH₂—, ═CHCH═CHCH₂O—, —CH═CHCH₂—, —CH═CHCH₂O—, —OCH₂CH₂O—, —CH₂—, —CH₂O—, —NHC(O)CH₂—, —NHC(O)CH₂O—, —C(O)CH₂—, —C(O)CH₂O—, —OC(O)CH₂—, OC(O)CH₂O—, —C(O)CH₂CH₂C(O)NHCH₂CH₂—, —OC(O)CH₂CH₂C(O)NHCH₂CH₂—, —C(O)CH₂CH₂C(O)NHCH₂CH₂O—, —OC(O)CH₂CH₂C(O)NHCH₂CH₂O—, —C(O)CH₂CH₂C(O)NHCH₂CH₂NHC(O)—, —OC(O)CH₂CH₂C(O)NHCH₂CH₂NHC(O)—, —C(O)CH₂CH₂C(O)NHCH₂CH₂NHC(O)O—, —OC(O)CH₂CH₂C(O)NHCH₂CH₂NHC(O)O—, or combinations thereof.

In some embodiments, the platinum based compounds are of Formula (I), wherein, X is NH; X₁ is —C(O)O—, —C(O)NH—, —O(CH₂)ₙO—, —NH—, or —O—; X₂ is —(CH₂)ₙ—, or C(O); X₃ is —(CH₂)ₙ—, —CH₂NH—, or C₄H₈; X₄ is C(O) or —CH(CH₃)—; Z is a platinum containing compound, wherein the platinum forms a part of Formula I ring; and n is 0, 1, or 2.

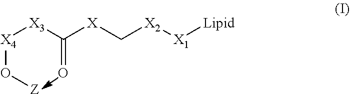

In other embodiments, the platinum based compounds are of Formula (II), wherein, X is NH or N—CH₂C(O)O⁻; X₁ is —(CH₂)ₙO—, —(CH₂)ₙNHC(O)O—, —(CH₂)ₙC(O)NH(CH₂)ₙO—, —(CH₂)ₙO(CH₂)ₙO—, —(CH₂)ₙC(O)—, —(CH₂)ₙNHC(O)(CH₂)ₙO—, or —(CH₂)ₙC(O)O—; Z is platinum containing compound, wherein the platinum forms a part of Formula II ring; and n is 0, 1, or 2.

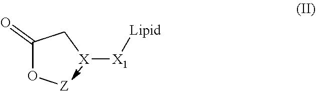

In further embodiments, the platinum based compounds are of Formula (III), wherein, X is S⁺, C, S⁺═O, N⁺H, or P═O; X₁ is —CH—, —CH₂— or —CH₂O—; X₂ is C(O); X₃ is —CH—, —CH₂—, or O; X₄, X₅, and X₆ are, independently, —CH₂O— or O; Z is platinum containing compound, wherein the platinum forms a part of Formula III ring.

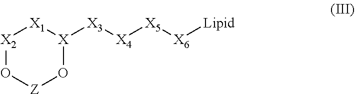

In yet other embodiments, the platinum based compounds are of Formula (IV), wherein, X is —CH₂O—; X₁ is —(CH₂)ₙ—; X₂ is C(O); Z is platinum containing compound, wherein the platinum forms a part of Formula IV ring; and n is 0, 1, or 2.

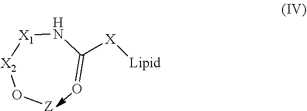

The compound of formula (VIII), including Compound 1, or pharmaceutically acceptable salts thereof, may also refer to any isomers thereof. As used herein, “isomer” refers to a compound having the same molecular formula, but different sequence of bonding or arrangement of the atoms. In some embodiments, the isomers are stereoisomers.

In some embodiments, the isomer includes one or more asymmetric centers, i.e., chiral centers, and thus includes diastereoisomers and enantiomers. The term “diastereoisomers” as used herein refer to stereoisomers that are not mirror images of each other and are non-superimposable. Similarly, the term “enantiomers” as used herein refer to stereoisomers that are mirror images of each other, but are non-superimposable. Thus, the relevant asymmetric center is described by the R- and S-sequencing rules of Cahn and Prelog. A chiral compound can exist as a single enantiomer or mixture thereof, i.e., a “racemic mixture.” The asymmetric center may also be described by the manner in which the molecule rotates the plane of polarized light and designated as dextrorotatory (+) or levorotatory (−).

In other aspects, the compound of formula (VIII), or pharmaceutically acceptable salts thereof, may be selected from among:

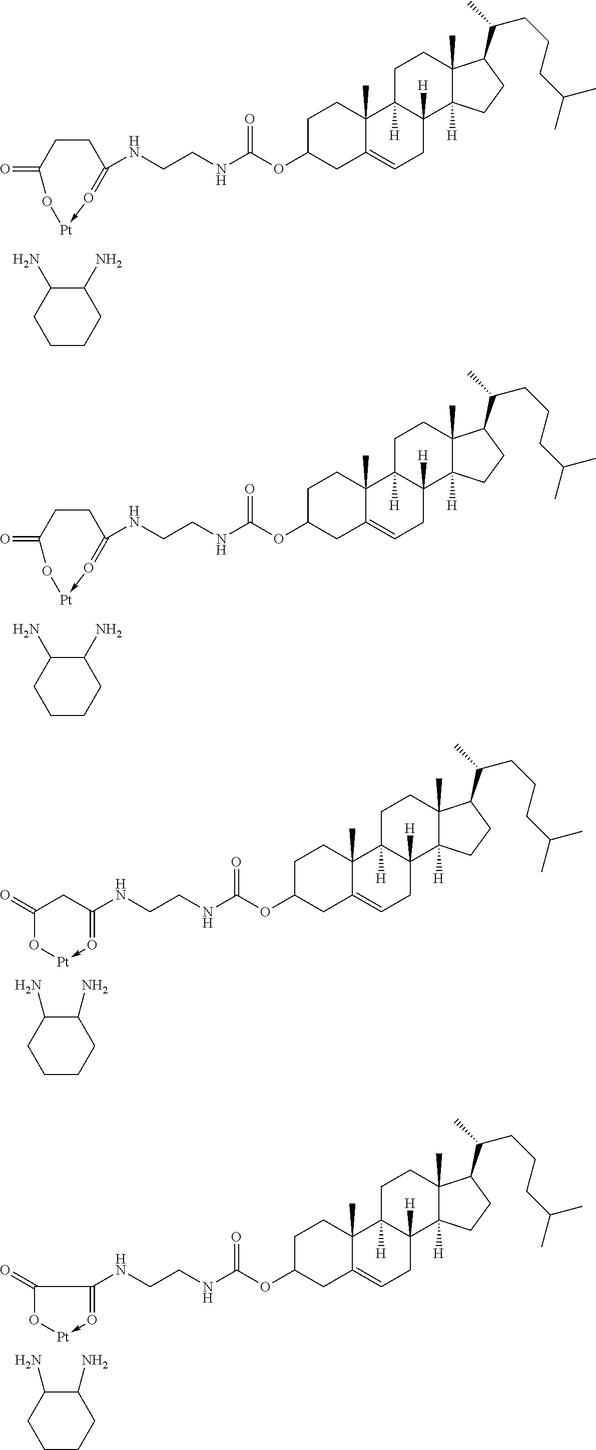

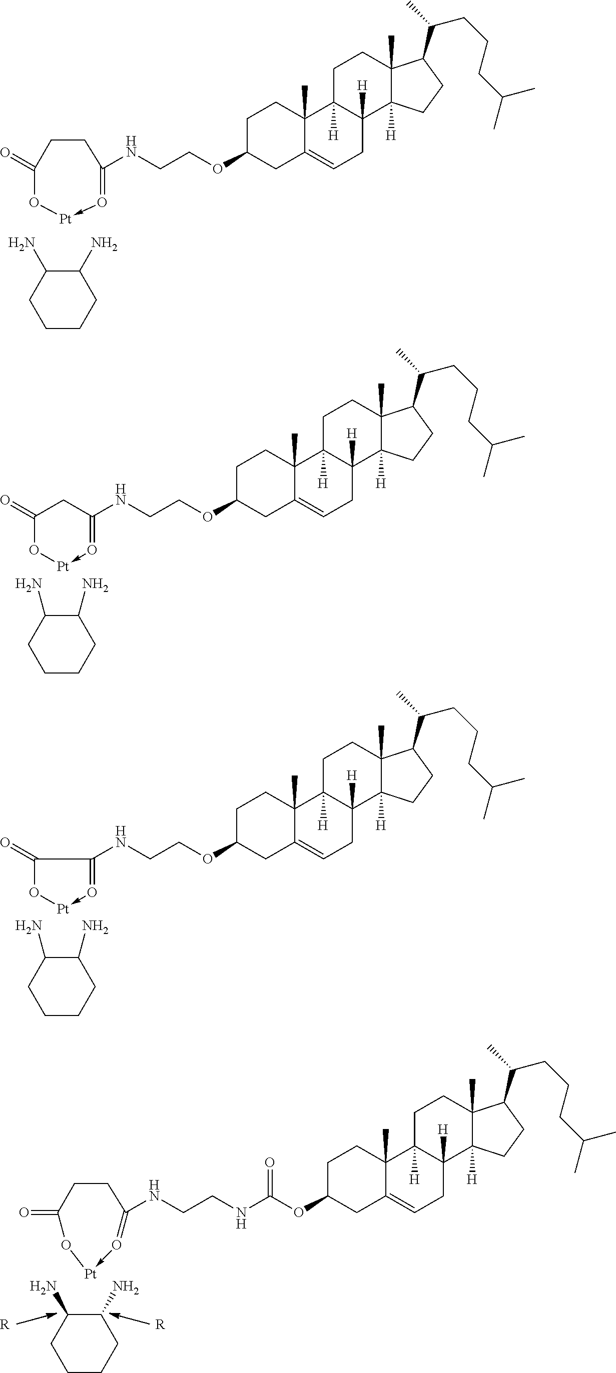

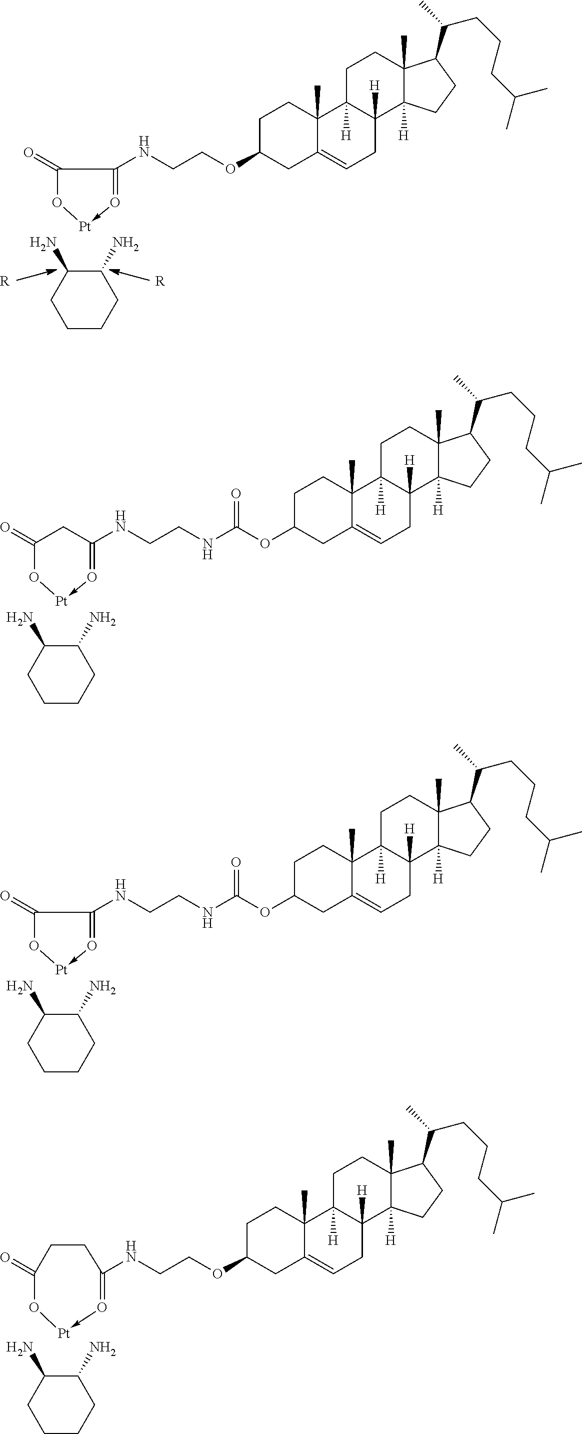

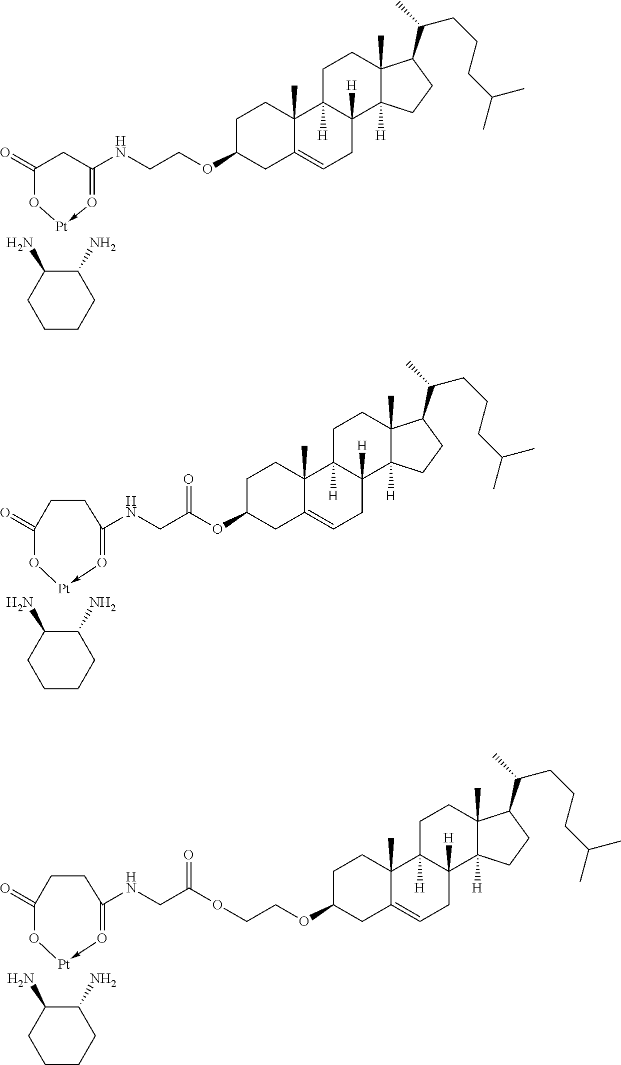

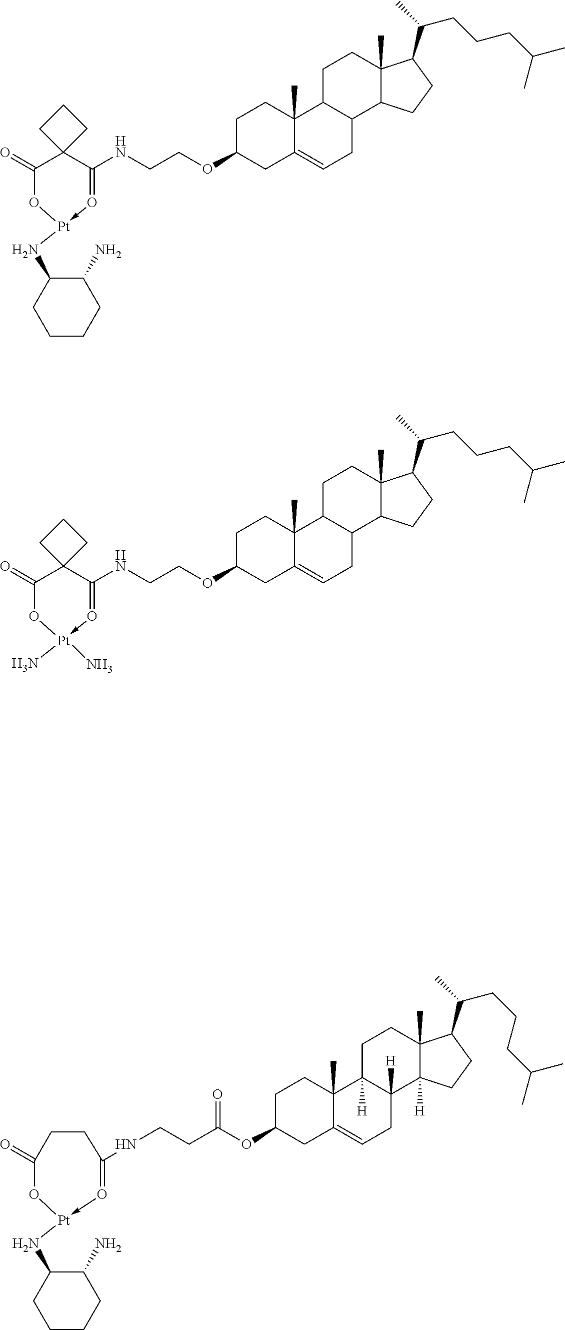

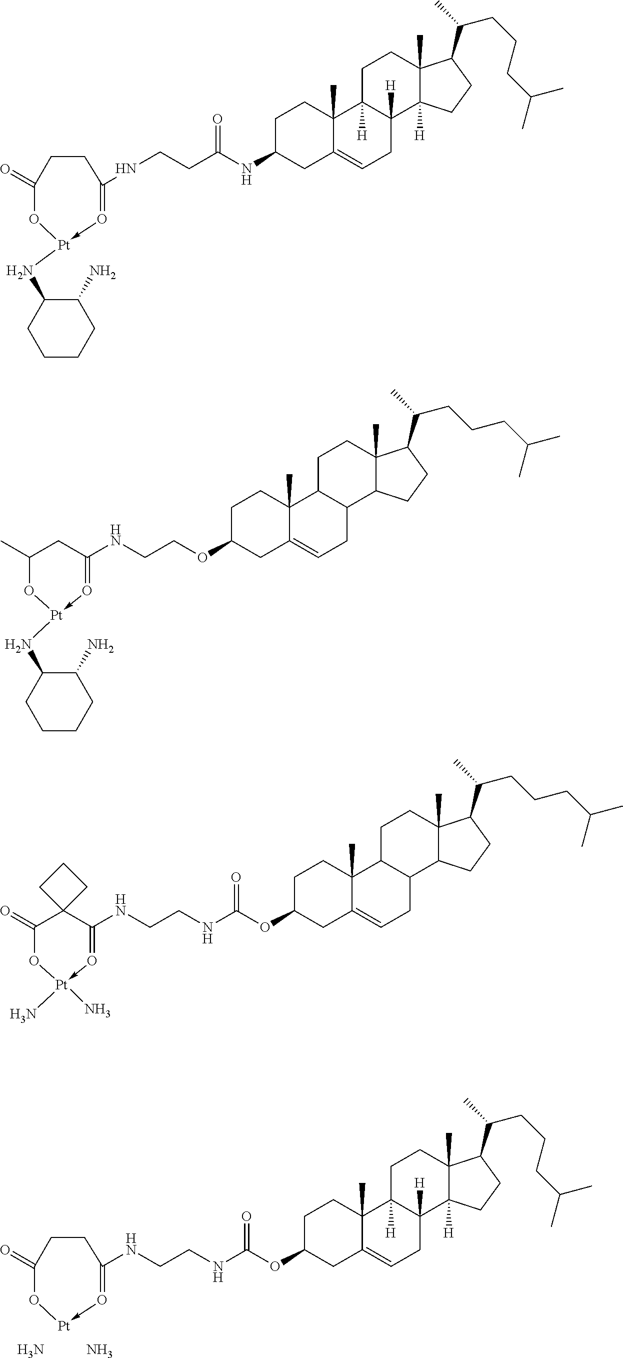

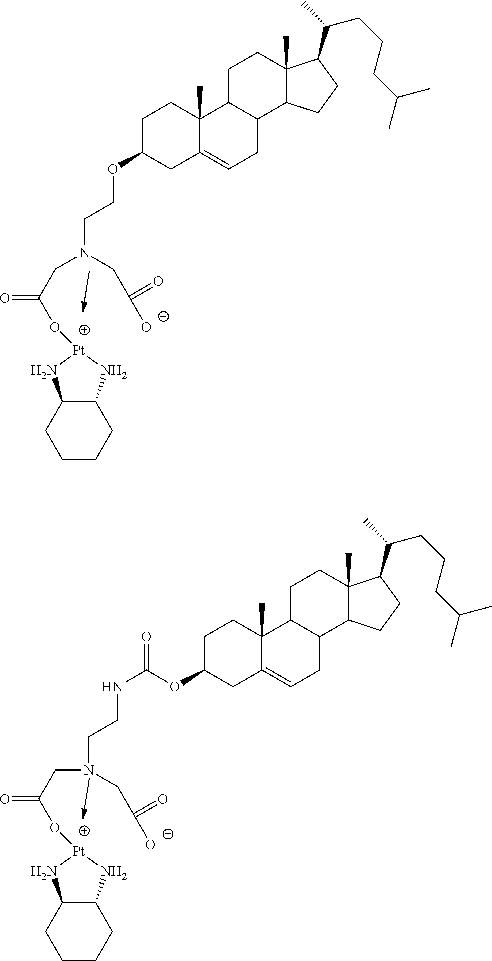

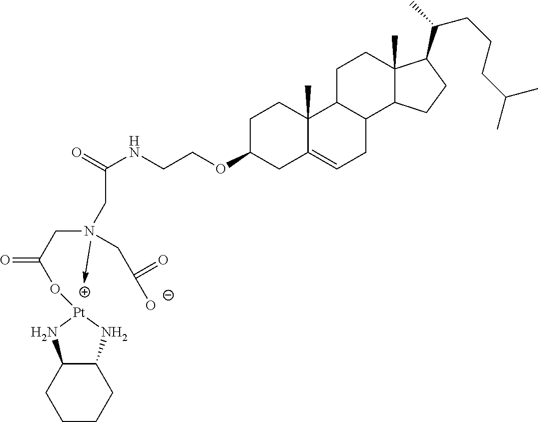

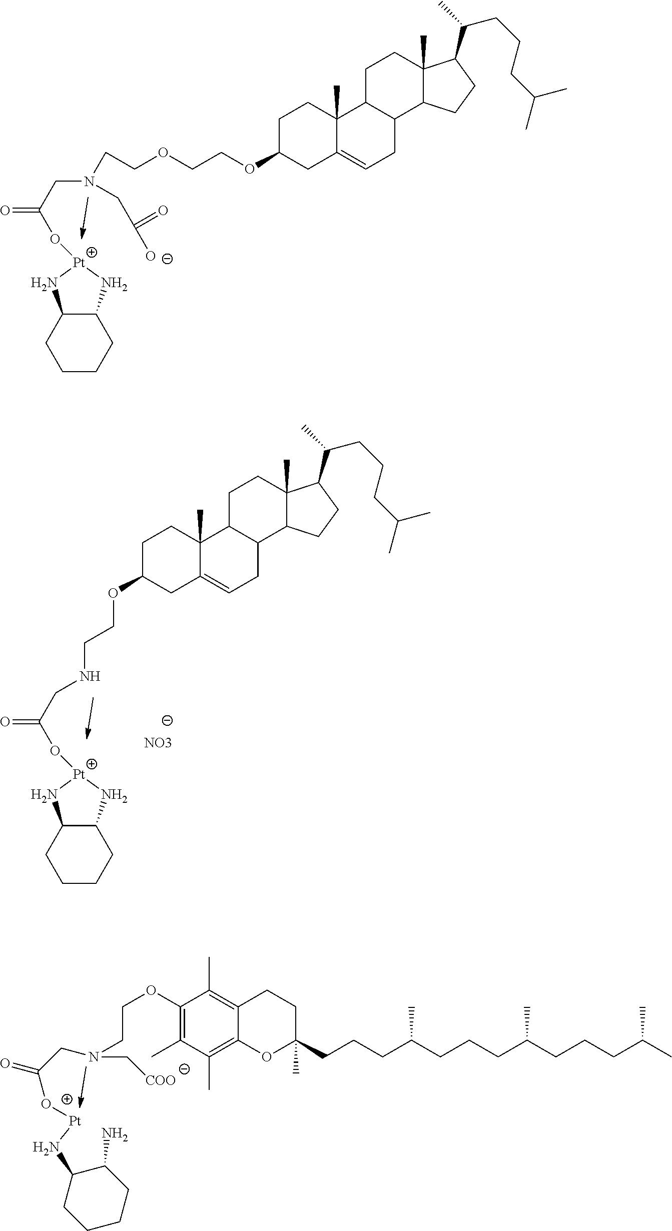

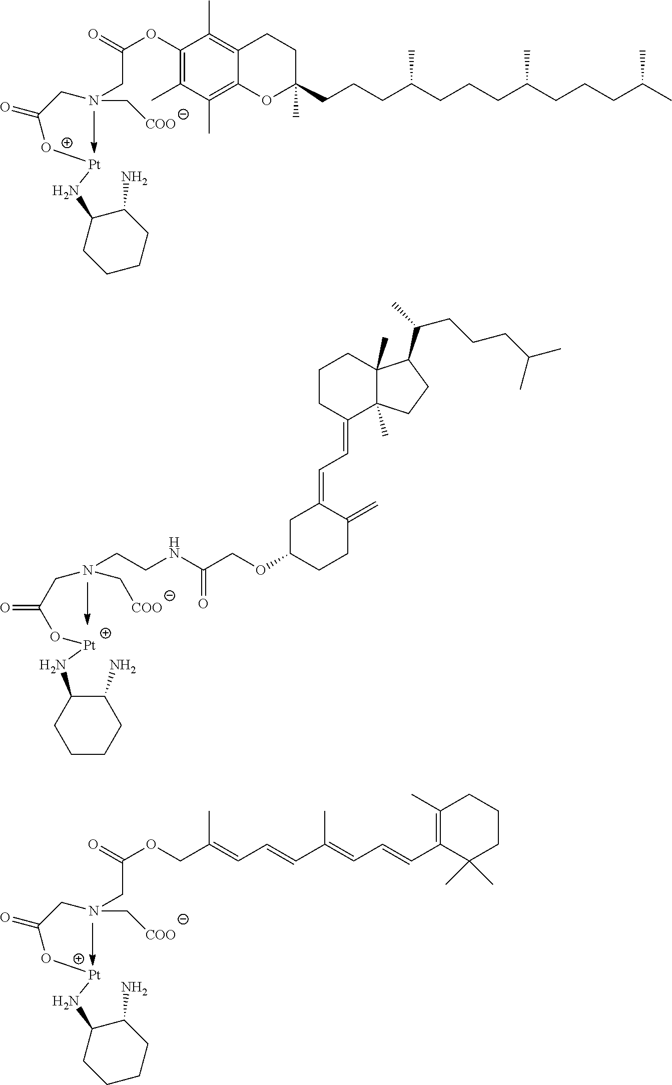

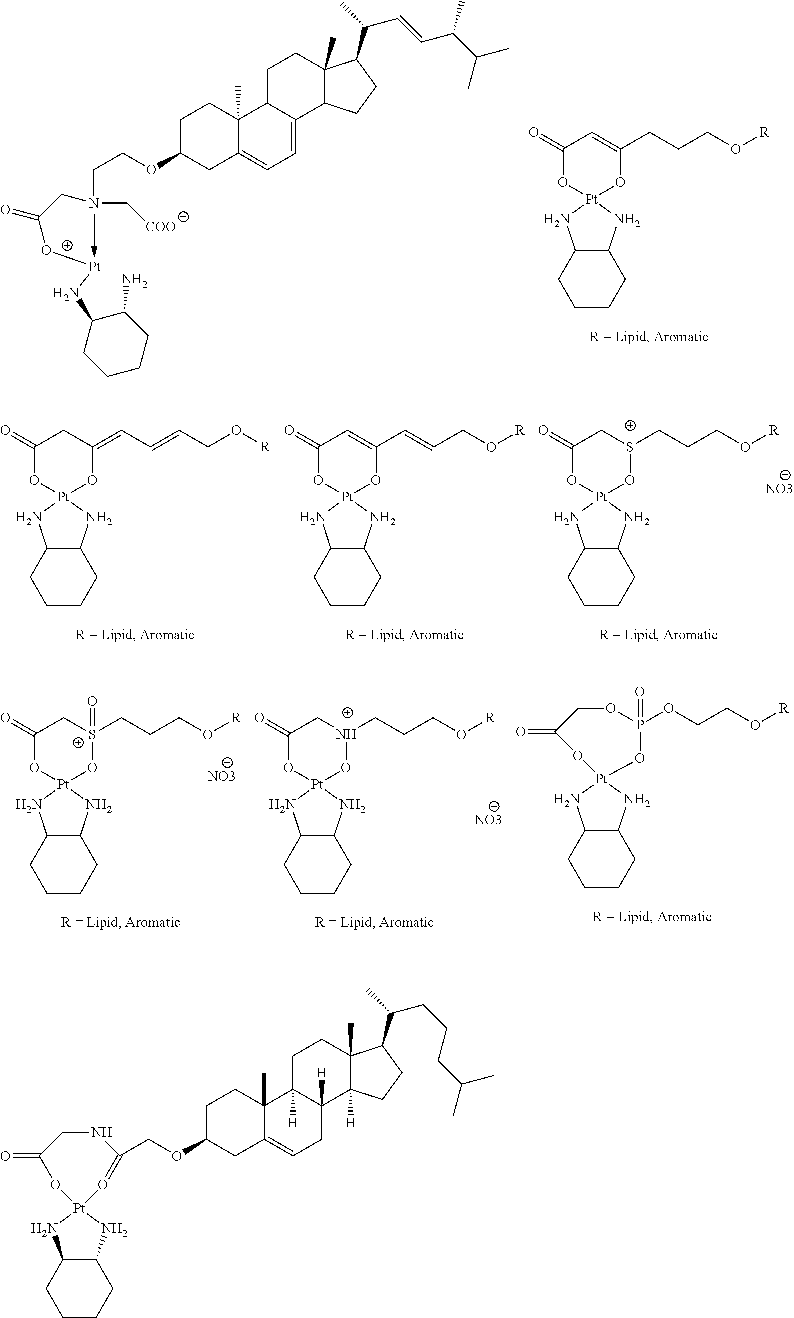

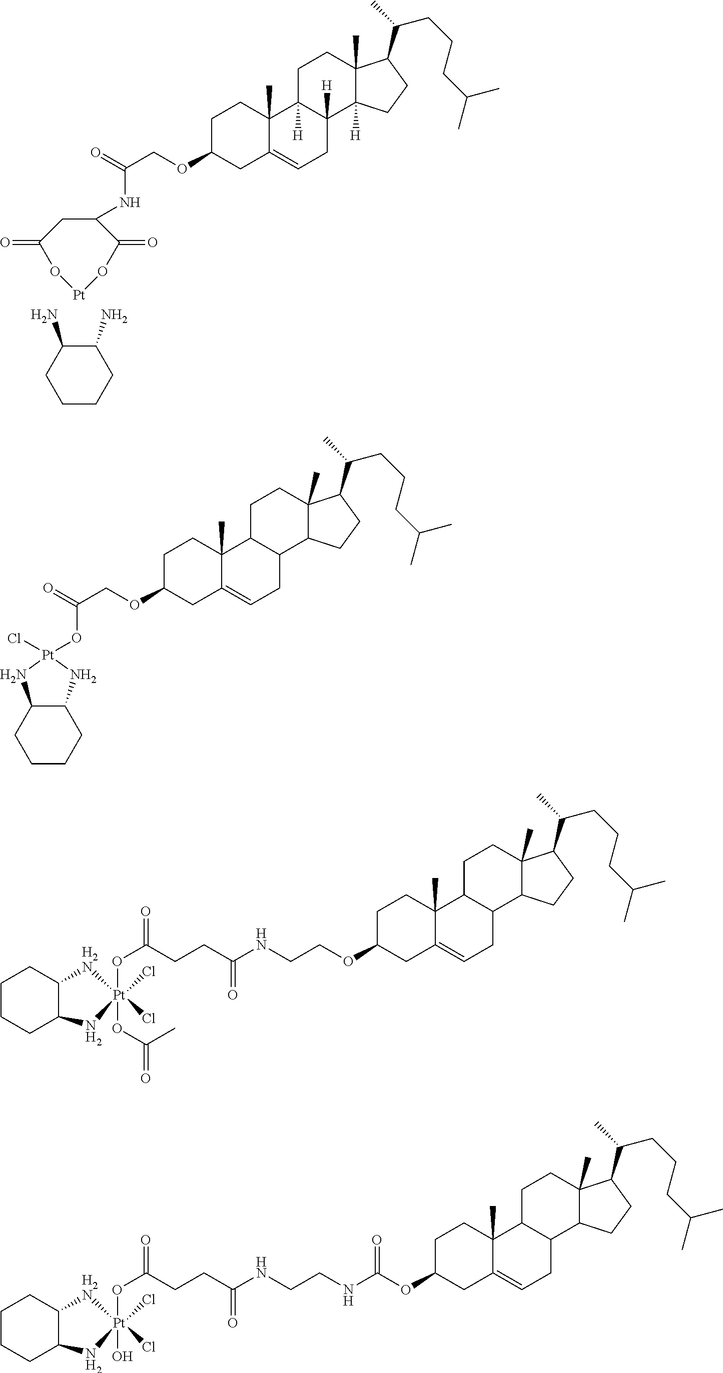

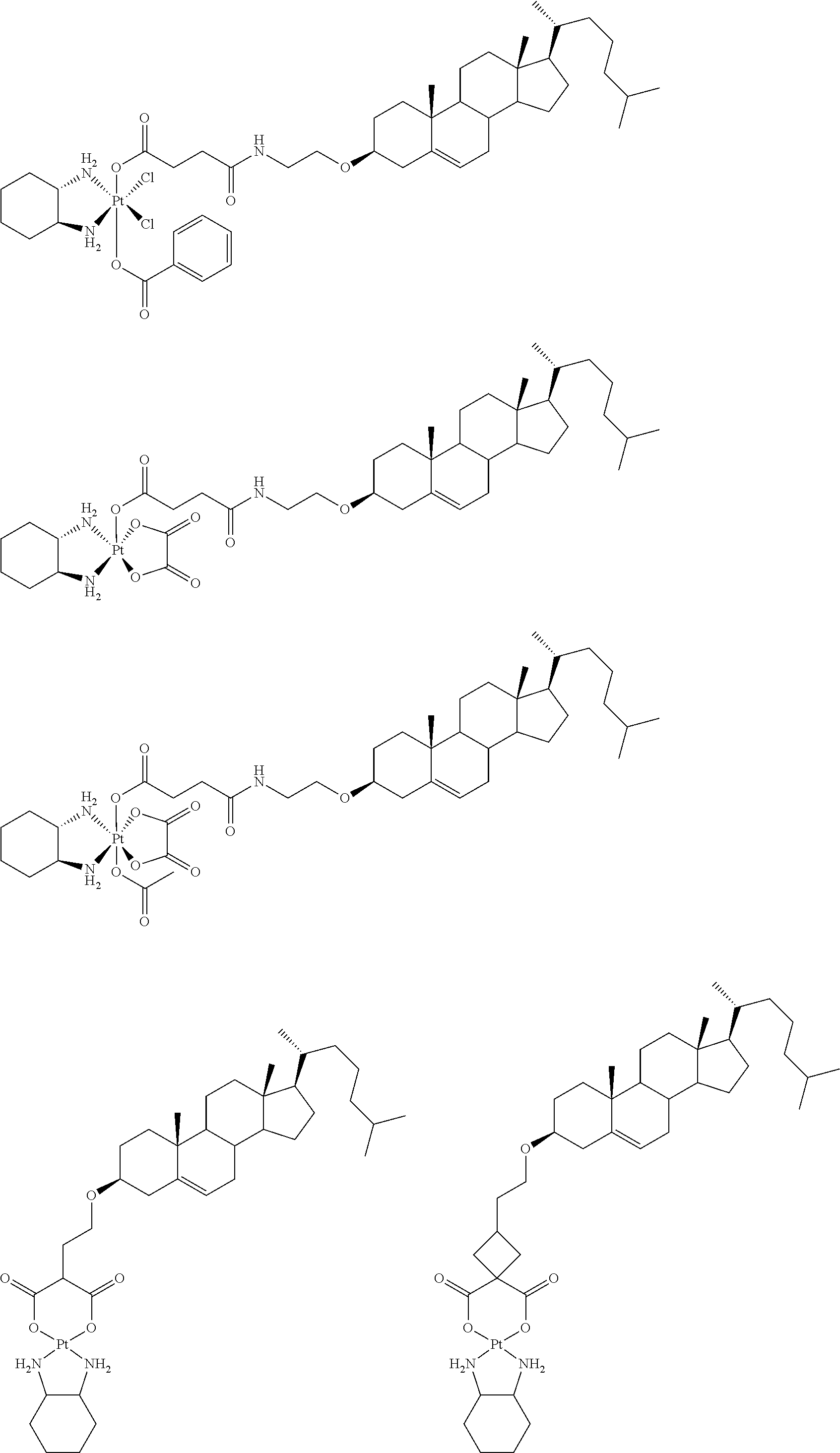

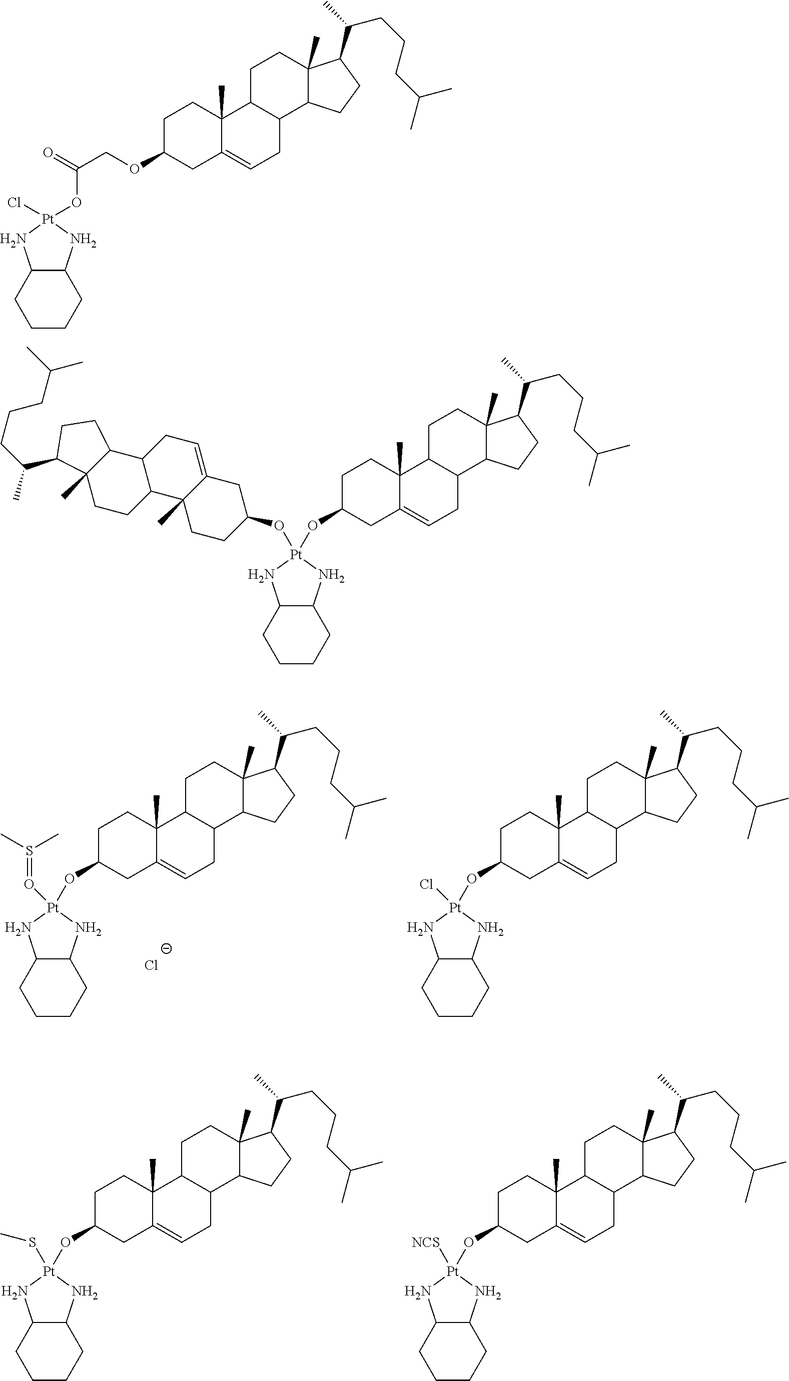

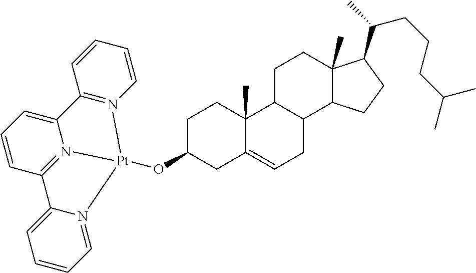

These compounds, including the compounds of formula (VIII), or pharmaceutically acceptable salts thereof, may be prepared as described in U.S. Patent Application Publication No. 2016/0145284, which is incorporated by reference herein.

In some aspects, the compound of formula (VIII) is Compound 1, as provided below:

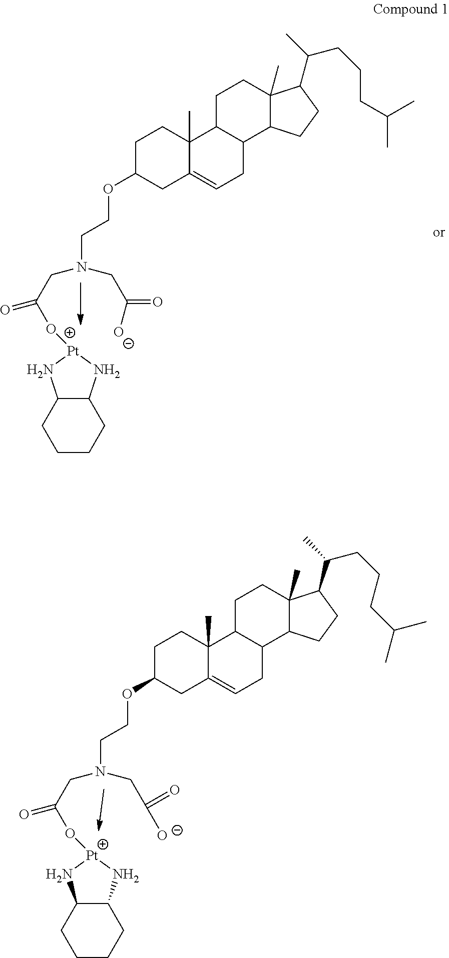

The synthesis of Compound 1, or pharmaceutically acceptable salts thereof, is described, for example, in PCT Application No. PCT/US2014/042339, published as WO/2014/201376, the content of which is incorporated herein by reference in its entirety.

In other embodiments, the disclosure provides the following compound A:

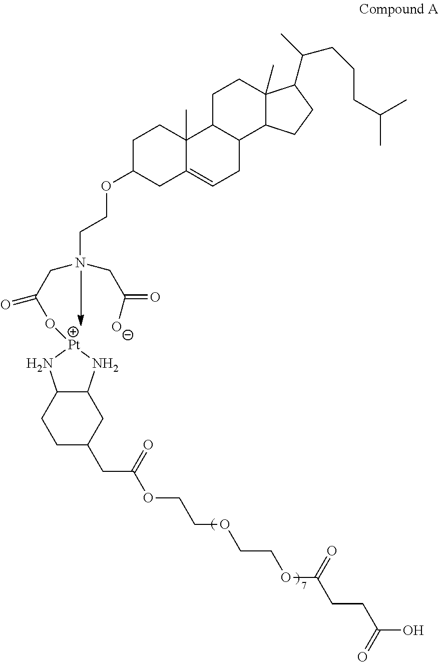

Further provided by the disclosure are stereoisomers of compound (A), or pharmaceutically acceptable salts thereof, such as enantiomers, diastereoisomers, among others. In some aspects, the disclosure provides a compound of formula (A-1):

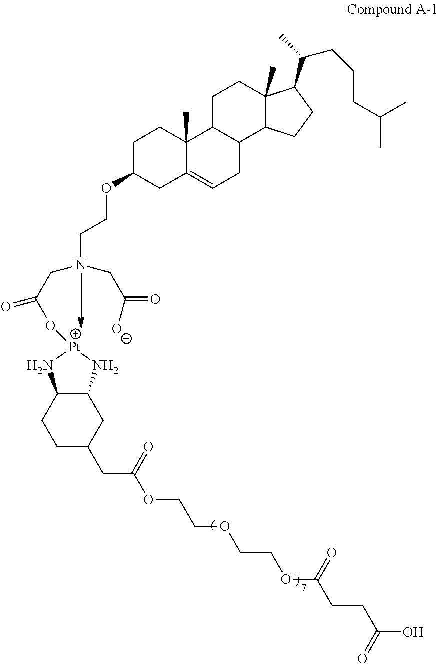

“Pharmaceutically acceptable salt” as used herein refers to salts of the compounds described herein, such compounds of formula (VIII), e.g., Compound 1, or Compound A or A-1 that are pharmaceutically acceptable and possess the activity of the neutral compound of formula (VIII), such as Compound 1. The salts are non-toxic and include inorganic acid, organic acid, or base addition salts. In some embodiments, the salts are inorganic acid salts. In other embodiments, the salts are formed with inorganic acids including, without limitation, hydrochloric acid, hydrobromic acid, sulfuric acid, nitric acid, or phosphoric acid. In further embodiments, the salts are formed using organic acids. In still other embodiments, the salts are formed with organic acids including, without limitation, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, or muconic acid. The pharmaceutically acceptable salts may also formed by replacing an acidic proton in the compounds of formula (VIII), such as Compound 1, with metal ion (alkali, alkaline earth, aluminum) or coordinates with an organic base (ethanolamine, diethanolamine, triethanolamine, N-methylglucamine).

The terms “subject” and “patient” are used interchangeably and include, without limitation, mammals. In some embodiments, the patient or subject is a human. In other embodiments, the patient or subject is a veterinary or farm animal, a domestic animal or pet, or animal used for clinical research. In some embodiments, the patient has cancer. In other embodiments, the patient previously had cancer and is in remission. In further embodiments, the patient has common variable immune deficiency (CVID), optionally associated with defective humoral immunity.

Thus, the present disclosure relates to methods of managing cancer by modulating/inhibiting cap methyltransferase enzyme by a compound described herein, such as the compound of formula (VIII), or pharmaceutically acceptable salts thereof. In some aspects, the compound of formula (VIII) is Compound 1, or pharmaceutically acceptable salts thereof. In other aspects, the compound is Compound A or A-1, or pharmaceutically acceptable salts thereof.

The present disclosure accordingly relates to use of the compounds described herein as inhibitors, such as compound of formula (VIII), or pharmaceutically acceptable salts thereof, for their use in modulating/inhibiting cap methyltransferase enzyme and management of cancer.

In some embodiments, the cancer is caused by B-cell depletion, T-cell depletion, or a combination thereof. In some aspects, the cancer is caused by B-cell depletion. In other aspects, the cancer is caused by T-cell depletion. In further aspects, the cancer is caused by B-cell and T-cell depletion. The cancer may be a solid cancer or hematological cancer. In some aspects, the cancer is a solid cancer. In other aspects, the cancer is a hematological cancer. In other embodiments, the cancer is prostate cancer, colorectal cancer, pancreatic cancer, cervical cancer, stomach cancer, endometrial cancer, brain cancer, liver cancer, bladder cancer, ovarian cancer, testicle cancer, head cancer, neck cancer, skin cancer such as melanoma or basal carcinoma, mesothelial lining cancer, white blood cell cancer such as lymphoma or leukaemia, esophageal cancer, breast cancer, muscle cancer, connective tissue cancer, lung cancer such as small-cell lung carcinoma or non-small-cell carcinoma, adrenal gland cancer, thyroid cancer, kidney cancer, or bone cancer. In other embodiments, the cancer is prostate cancer. In further embodiments, the cancer is colorectal cancer. In other embodiments, the cancer is pancreatic cancer. In still further embodiments, the cancer is cervical cancer. In other embodiments, the cancer is cervical epithelial cancer. In further embodiments, the cancer is stomach cancer. In other embodiments, the cancer is endometrial cancer. In yet further embodiments, the cancer is brain cancer. In still other embodiments, the cancer is liver cancer. In further embodiments, the cancer is bladder cancer. In other embodiments, the cancer is ovarian cancer. In still further embodiments, the cancer is testicle cancer. In yet other embodiments, the cancer is head cancer. In further embodiments, the cancer is neck cancer. In other embodiments, the cancer is skin cancer. In yet further embodiments, the cancer is skin cancer such as melanoma or basal carcinoma. In still other embodiments, the cancer is mesothelial lining cancer. In further embodiments, the cancer is a white blood cell cancer. In other embodiments, the cancer is a white blood cell cancer such as lymphoma or leukaemia, preferably lymphoma. In yet further embodiments, the cancer is esophageal cancer. In still other embodiments, the cancer is breast cancer. In other embodiments, the cancer is triple negative breast cancer (TNBC) or luminal B-type breast cancer. In further embodiments, the cancer is muscle cancer. In other embodiments, the cancer is connective tissue cancer. In yet further embodiments, the cancer is lung cancer. In still other embodiments, the cancer is lung cancer such as small-cell lung carcinoma or non-small-cell carcinoma. In further embodiments, the cancer is adrenal gland cancer. In other embodiments, the cancer is thyroid cancer. In still further embodiments, the cancer is kidney cancer. In yet other embodiments, the cancer is bone cancer.

A therapeutically effective amount of a compound described herein, such as the compound of formula (VIII), e.g., Compound 1, or Compound A or A-1, or salts thereof is administered to a subject suffering from or diagnosed as having cancer. As used herein, a “therapeutically effective amount” refers to an amount or dose sufficient to reduce or ameliorate cancer cells in a patient. Therapeutically effective amounts may be determined by those skilled in the art, such as an attending physician, using modeling, dose escalation studies or clinical trials. In some embodiments, the therapeutically effective amount of the compound described herein, such as the compound of formula (VIII), e.g., Compound 1, or Compound A or A-1, or pharmaceutically acceptable salts thereof, is in the range of from about 0.001 to about 200 mg of compound per kg of subject's body weight per day. In other embodiments, the therapeutically effective amount is about 0.05 to about 100 mg/kg/day. In further embodiments, the therapeutically effective amount is about 1 to about 35 mg/kg/day. In yet other embodiments, the therapeutically effective amount is about 1 to about 30, about 1 to about 25, about 1 to about 20, about 1 to about 15, about 1 to about 10, about 1 to about 5, about 5 to about 35, about 5 to about 30, about 5 to about 25, about 5 to about 20, about 5 to about 15, about 5 to about 10, about 10 to about 35, about 10 to about 30, about 10 to about 25, about 10 to about 20, about 10 to about 15, about 15 to about 35, about 15 to about 30, about 15 to about 25, about 15 to about 20, about 20 to about 35, about 20 to about 30, or about 25 to about 35 mg/kg/day. By way of example, a 70-kg human, an illustrative range for a dose of a compound described herein, such as the compound of formula (VIII), e.g., Compound 1, Compound A or A-1, or salts thereof is from about 0.001 to about 7, about 0.1 to about 7, about 0.5 to about 7, about 1 to about 7, about 2 to about 7, about 3 to about 7, about 4 to about 7, about 5 to about 7, about 0.1 to about 5, about 0.1 to about 4, about 0.1 to about 3, about 0.1 to about 2, about 0.2 to about 7, about 0.2 to about 6, about 0.2 to about 5, about 0.2 to about 4, about 0.2 to about 3, or about 0.2 to about 2.5 g/day.

Alternatively, the therapeutically effective amount is about 0.001 to about 500 mg/kg/day. In yet other embodiments, the therapeutically effective amount is about 0.001 to about 400, about 0.001 to about 300, about 0.001 to about 200, about 0.005 to about 400, about 0.005 to about 300, about 0.005 to about 200, about 0.010 to about 400, about 0.010 to about 300, about 0.010 to about 200, about 0.05 to about 400, about 0.05 to about 300, about 0.05 to about 200, about 0.1 to about 400, about 0.1 to about 300, about 0.1 to about 200, about 0.5 to about 400, about 0.5 to about 300, or about 0.5 to about 200 mg/kg/day. By way of example, a 70-kg human, an illustrative range for a dose of a compound described herein, such as the compound of formula (VIII), e.g., Compound 1, Compound A or A-1, or salt thereof is from about 0.00007 to about 28, about 0.00007 to about 21, about 0.00007 to about 14, about 0.00035 to about 28, about 0.00035 to about 21, about 0.00035 to about 14, about 0.0007 to about 28, about 0.0007 to about 21, about 0.0007 to about 14, about 0.0035 to about 28, about 0.0035 to about 21, about 0.0035 to about 14, about 0.007 to about 28, about 0.007 to about 21, about 0.007 to about 14, about 0.035 to about 28, about 0.035 to about 21, or about 0.035 to about 14 g/day.

The therapeutically effective amount may be administered in single or divided dosage units.

The disclosure also provides nanoparticles comprising one or more of the platinum based compounds, compounds of formula (VIII), Compound 1, Compound A, or Compound A-1, or pharmaceutically acceptable salts thereof, described herein. In some embodiments, the nanoparticles comprise Compound 1. Generally, the nanoparticles disclosed herein can be of any shape or form, e.g., spherical, rod, elliptical, cylindrical, capsule, or disc; and these particles can be part of a network or an aggregate.

As used the term “nanoparticle” refers to particle having a particle size of about 0.1 nm to about 1000 nm. In some embodiments, the nanoparticles can have a size ranging from about 5 nm to about 1000 nm. In other embodiments, the nanoparticles have an average diameter of from about 50 nm to about 1000 nm. In further embodiments, the nanoparticles have an average diameter of from about 100 nm to about 1000 nm. In yet other embodiments, the nanoparticles have an average diameter of from about 150 nm to about 1000 nm. In still further embodiments, the nanoparticles have an average diameter of from about 200 nm to about 1000 nm. In other embodiments, the nanoparticles have an average diameter of about 260 nm. In further embodiments, the nanoparticles have an average diameter of about 30 nm to about 150 nm. In still other embodiments, the nanoparticle have an average diameter of about 200 nm to about 800 nm. In yet further embodiments, the nanoparticles have an average diameter of about 200 nm to about 700 nm. In other embodiments, the nanoparticles have an average size of about 300 nm to about 700 nm. In further embodiments, the nanoparticles have an average size of 50 to about 500 nm. In yet other embodiments, the nanoparticles have a size of about 500 nm.

It will be understood by one of ordinary skill in the art that nanoparticles usually exhibit a distribution of particle sizes around the indicated “size.” Unless otherwise stated, the term “particle size” as used herein refers to the mode of a size distribution of nanoparticles, i.e., the value that occurs most frequently in the size distribution. Methods for measuring the particle size are known to a skilled artisan, e.g., by dynamic light scattering (such as photocorrelation spectroscopy, laser diffraction, low-angle laser light scattering (LALLS), and medium-angle laser light scattering (MALLS)), light obscuration methods (such as Coulter analysis method), or other techniques (such as rheology, and light or electron microscopy).

In some embodiments, the nanoparticles are substantially spherical. The term “substantially spherical” is that the ratio of the lengths of the longest to the shortest perpendicular axes of the particle cross section is less than or equal to about 1.5. Substantially spherical does not require a line of symmetry. Further, the particles can have surface texturing, such as lines or indentations or protuberances that are small in scale when compared to the overall size of the particle and still be substantially spherical. In some embodiments, the ratio of lengths between the longest and shortest axes of the particle is less than or equal to about 1.5, less than or equal to about 1.45, less than or equal to about 1.4, less than or equal to about 1.35, less than or equal to about 1.30, less than or equal to about 1.25, less than or equal to about 1.20, less than or equal to about 1.15 less than or equal to about 1.1. Without wishing to be bound by a theory, surface contact is minimized in particles that are substantially spherical, which minimizes the undesirable agglomeration of the particles upon storage. Many crystals or flakes have flat surfaces that can allow large surface contact areas where agglomeration can occur by ionic or non-ionic interactions. A sphere permits contact over a much smaller area.

In some embodiments, the nanoparticles have substantially the same particle size. Nanoparticles having a broad size distribution where there are both relatively big and small particles allow for the smaller particles to fill in the gaps between the larger particles, thereby creating new contact opportunities for binding agglomeration. The nanoparticles described herein are within a narrow size distribution, thereby minimizing opportunities for contact agglomeration. The phrase “narrow size distribution” refers to a particle size distribution that has a ratio of the volume diameter of the 90th percentile of the small spherical particles to the volume diameter of the 10th percentile less than or equal to 5. In some embodiments, the volume diameter of the 90th percentile of the small spherical particles to the volume diameter of the 10th percentile is less than or equal to 4.5, less than or equal to 4, less than or equal to 3.5, less than or equal to 3, less than or equal to 2.5, less than or equal to 2, less than or equal to 1.5, less than or equal to 1.45, less than or equal to 1.40, less than or equal to 1.35, less than or equal to 1.3, less than or equal to 1.25, less than or equal to 1.20, less than or equal to 1.15, or less than or equal to 1.1.

The nanoparticle can also comprise lipids and/stabilizers. Any of a number of additional lipids and/or other components can be present, including amphipathic, neutral, cationic, anionic lipids, and programmable fusion lipids. Such lipids and/or components can be used alone or in combination. One or more components of the nanoparticles can comprise a ligand, e.g., a targeting ligand. In some embodiments, the nanoparticles comprise one lipid. In other embodiments, the nanoparticles comprise two lipids.

In some embodiments, the lipid is a phospholipid. Without limitation, the phospholipids can be of natural origin, such as egg yolk or soybean phospholipids, or synthetic or semisynthetic origin. The phospholipids can be partially purified or fractionated to comprise pure fractions or mixtures of phosphatidyl cholines, phosphatidyl cholines with defined acyl groups having 6 to 22 carbon atoms, phosphatidyl ethanolamines, phosphatidyl inositols, phosphatidic acids, phosphatidyl serines, sphingomyelin or phosphatidyl glycerols.

Suitable phospholipids include, but are not limited to, phosphatidylcholine, L-α-phosphatidylcholine, hydrogenated (soy), phosphatidylglycerol, lecithin, β,γ-dipalmitoyl-α-lecithin, sphingomyelin, phosphatidylserine, phosphatidic acid, N-(2,3-di(9-(Z)-octadecenyloxy))-prop-1-yl-N,N,N-trimethylammonium chloride, phosphatidylethanolamine, lysolecithin, lysophosphatidylethanolamine, phosphatidylinositol, cephalin, cardiolipin, cerebrosides, dicetylphosphate, dioleoylphosphatidylcholine, dipalmitoylphosphatidylcholine, dipalmitoylphosphatidylglycerol, dioleoylphosphatidylglycerol, palmitoyl-oleoyl-phosphatidylcholine, di-stearoylphosphatidylcholine, stearoyl-palmitoyl-phosphatidylcholine, di-palmitoylphosphatidylethanolamine, di-stearoyl-phosphatidylethanolamine, di-myrstoylphosphatidylserine, di-oleyl-phosphatidylcholine, dimyristoyl phosphatidyl choline (DMPC), dioleoylphosphatidylethanolamine (DOPE), palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), -phosphatidylethanolamine (POPE), dioleoylphosphatidylethanolamine 4-(N-maleimidomethyl)-cyclohexane-1-carboxylate (DOPE-mal), 1-stearoyl-2-oleoyl phosphatidylcholine (SOPC), 1,2-distearoyl-sn-glycero-3-phosphoethanolamine (DSPE), 1,2-distearoyl-sn-glycero-3-phosphoethanolamine (DSPE) ammonium salt, and any combinations thereof. Non-phosphorus containing lipids can also be used. These include, e.g., stearylamine, docecylamine, acetyl palmitate, fatty acid amides, and the like. Other phosphorus-lacking compounds, such as sphingolipids, glycosphingolipid families, diacylglycerols, and β-acyloxyacids, can also be used. In some embodiments, the phospholipid in the nanoparticle is 1,2-didecanoyl-sn-glycero-3-phosphocholine; 1,2-dierucoyl-sn-glycero-3-phosphate (sodium salt); 1,2-dierucoyl-sn-glycero-3-phosphocholine; 1,2-dierucoyl-sn-glycero-3-phosphoethanolamine; 1,2-dierucoyl-sn-glycero-3[phospho-rac-(1-glycerol) (sodium salt); 1,2-dilinoleoyl-sn-glycero-3-phosphocholine; 1,2-dilauroyl-sn-glycero-3-phosphate (sodium salt); 1,2-dilauroyl-sn-glycero-3-phosphocholine; 1,2-dilauroyl-sn-glycero-3-phosphoethanolamine; 1,2-dilauroyl-sn-glycero-3[phospho-rac-(1-glycerol) (sodium salt); 1,2-dilauroyl-sn-glycero-3[phospho-rac-(1-glycerol) (ammonium salt); 1,2-dilauroyl-sn-glycero-3-phosphoserine (sodium salt); 1,2-dimyristoyl-sn-glycero-3-phosphate (sodium salt); 1,2-dimyristoyl-sn-glycero-3-phosphocholine; 1,2-dimyristoyl-sn-glycero-3-phosphoethanolamine; 1,2-dimyristoyl-snglycero-3[phospho-rac-(1-glycerol) (sodium salt); 1,2-dimyristoyl-sn-glycero-3[phospho-rac-(1-glycerol) (ammonium salt); 1,2-dimyristoyl-sn-glycero-3[phospho-rac-(1-glycerol) (sodium/ammonium salt); 1,2-dimyristoyl-sn-glycero-3-phosphoserine (sodium salt); 1,2-dioleoyl-sn-glycero-3-phosphate (sodium salt); 1,2-dioleoyl-sn-glycero-3-phosphocholine; 1,2-dioleoyl-sn-glycero-3-phosphoethanolamine; 1,2-dioleoyl-sn-glycero-3[phospho-rac-(1-glycerol)(sodium salt); 1,2-dioleoyl-sn-glycero-3-phosphoserine (sodium salt); 1,2-dipalmitoyl-snglycero-3-phosphate (sodium salt); 1,2-dipalmitoyl-sn-glycero-3-phosphocholine; 1,2-dipalmitoyl-sn-glycero-3-phosphoethanolamine; 1,2-dipalmitoyl-sn-glycero-3[phospho-rac-(1-glycerol) (sodium salt); 1,2-dipalmitoyl-sn-glycero-3[phospho-rac-(1-glycerol) (ammonium salt); 1,2-dipalmitoyl-sn-glycero-3-phosphoserine (sodium salt); 1,2-distearoyl-sn-glycero-3-phosphate (sodium salt); 1,2-distearoyl-sn-glycero-3-phosphocholine; 1,2-distearoyl-sn-glycero-3-phosphoethanolamine; 1,2-distearoyl-sn-glycero-3[phospho-rac-(1-glycerol) (sodium salt); 1,2-distearoyl-sn-glycero-3[phospho-rac-(1-glycerol) (ammonium salt); 1,2-distearoyl-snglycero-3-phosphoserine (sodium salt); Egg-PC; Hydrogenated Egg PC; hydrogenated soy PC (L-α-phosphatidylcholine, hydrogenated (soy)); 1-myristoyl-sn-glycero-3-phosphocholine; 1-palmitoyl-sn-glycero-3-phosphocholine; 1-stearoylsn-glycero-3-phosphocholine; 1-myristoyl-2-palmitoyl-sn-glycero 3-phosphocholine; 1-myristoyl-2-stearoyl-sn-glycero-3-phosphocholine; 1-palmitoyl-2-myristoyl-sn-glycero-3-phosphocholine; 1-palmitoyl-2-oleoyl-sn-glycero-3-phosphocholine; 1-palmitoyl-2-oleoyl-snglycero-3-phosphoethanolamine; 1-palmitoyl-2-oleoyl-sn-glycero-3[phospho-rac-(1-glycerol)] (sodium salt); 1-palmitoyl-2-stearoyl-sn-glycero-3-phosphocholine; 1-stearoyl-2-myristoyl-snglycero-3-phosphocholine; 1-stearoyl-2-oleoyl-sn-glycero-3-phosphocholine; and 1-stearoyl-2-palmitoyl-sn-glycero-3-phosphocholine. In some embodiments, the phospholipid is SPOC, egg PC, or Hydrogenated Soy PC (HSPC). In one, the phospholipid in the composition is HSPC. In further embodiments, the phospholipid is L-α-phospatidylcholine, hydrogenated (soy).

In other embodiments, the lipid a polyethylene glycol (PEG). The PEG can be included in the nanoparticle by itself or conjugated with a component present in the nanoparticle. For example, the PEG can be conjugated with the platinum based compound or a colipid/stabilizer component of the nanoparticle. In some embodiments, the PEG is conjugated with a co-lipid component of the nanoparticle. Without limitation, the PEG can be conjugated with any colipid. For example, the PEG conjugated co-lipid is a PEG conjugated diacylglycerols and dialkylglycerols, PEG-conjugated phosphatidylethanolamine, PEG conjugated to phosphatidic acid, PEG conjugated ceramides (see, U.S. Pat. No. 5,885,613), PEG conjugated dialkylamines, PEG conjugated 1,2-diacyloxypropan-3-amines, or PEG conjugated to 1,2-distearoyl-sn-glycero-3-phosphoethanolamine (DSPE), or any combinations thereof. In some embodiments, the PEG conjugated lipid is 1,2-distearoyl-sn-glycero-3-phosphoethanolamine-N-[methoxy(polyethylene glycol)-2000] (DSPE-PEG2000). In other embodiments, the PEG conjugated lipid is 1,2-distearoyl-sn-glycero-3-phosphoethanolamine-N-[methoxy(polyethylene glycol)-2000] (ammonium salt).

In further embodiments, the nanoparticles comprise two lipids (co-lipids). The co-lipids comprise is 1,2-distearoyl-sn-glycero-3-phosphoethanolamine-N-[methoxy(polyethylene glycol)-2000] (ammonium salt) and L-α-phospatidylcholine, hydrogenated (soy).

In some embodiments, the nanoparticles comprise a surfactant. Examples of surfactants include nonionic surfactants, anionic surfactants, cationic surfactants, or amphoteric surfactants. If the surfactant is not ionized, it is classified as a nonionic surfactant. on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The nanoparticle may also comprise a lipid. In some aspects, the lipid is a cationic lipid, anionic lipid, or neutral lipid.

In some embodiments, the lipid is a cationic lipid. Exemplary cationic lipids include, but are not limited to, N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), N,N-dimethyl-2,3-dioleyloxy)propylamine (DODMA), 1,2-diLinoleyloxy-N,N-dimethylaminopropane (DLinDMA), 1,2-dilinolenyloxy-N,N-dimethylaminopropane (DLenDMA), 1,2-dilinoleylcarbamoyloxy-3-dimethylaminopropane (DLin-C-DAP), 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-Dilinoleyloxy-3-(N-methylpiperazino)propane (DLin-MPZ), or 3-(N,N-dilinoleylamino)-1,2-propanediol (DLinAP), 3-(N,N-dioleylamino)-1,2-propanedio (DOAP), 1,2-dilinoleyloxo-3-(2-N,Ndimethylamino)ethoxypropane (DLin-EG-DMA), 1,2-dilinolenyloxy-N,Ndimethylaminopropane (DLinDMA), 2,2-dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta[d][1,3]dioxol-5-amine (ALN100), (6Z,9Z,28Z,31Z)-heptatriaconta-6,9,28,31-tetraen-19-yl 4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (Tech Gi), or a mixture thereof.

In other embodiments, the lipid is an anionic lipid or a neutral lipid. Examples of such lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPEmal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof.

Conjugated lipids that inhibit aggregation of particles can also be included in the nanoparticles disclosed herein. Such lipids include, but are not limited to, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate can be, for example, a PEG-dilauryloxypropyl (C12), a PEG-dimyristyloxypropyl (C14), a PEGdipalmityloxypropyl (C16), or a PEG-distearyloxypropyl (C18). The conjugated lipid that prevents aggregation of particles can be from 0.01 mol % to about 20 mol % or about 2 mol % of the total lipid present in the nanoparticles.

The nanoparticles may also be in the form of a liposome, vesicle, or emulsion. As used herein, the term “liposome” encompasses any compartment enclosed by a lipid layer. Liposomes can have one or more lipid membranes. Liposomes can be characterized by membrane type and by size. Small unilamellar vesicles (SUVs) have a single membrane and typically range between 0.02 and 0.05 μm in diameter; large unilamellar vesicles (LUVS) are typically larger than 0.05 μm. Oligolamellar large vesicles and multilamellar vesicles have multiple, usually concentric, membrane layers and are typically larger than 0.1 μm. Liposomes with several nonconcentric membranes, i.e., several smaller vesicles contained within a larger vesicle, are termed multivesicular vesicles.

Liposome compositions comprising the nanoparticle can be prepared by a variety of methods that are known in the art. See, e.g., U.S. Pat. Nos. 4,235,871; 4,897,355; and 5,171,678; International Patent Publication No. WO-96/14057 and WO 96/37194; Felgner, Proc. Natl. Acad. Sci., USA (1987) 8:7413-7417, Bangham, M. Mol. Biol. (1965) 23:238, Olson, Biochim. Biophys. Acta (1979) 557:9, Szoka, Proc. Natl. Acad. Sci. (1978) 75: 4194, Mayhew, Biochim. Biophys. Acta (1984) 775:169, Kim, Biochim. Biophys. Acta (1983) 728:339, and Fukunaga, Endocrinol. (1984) 115:757. The liposomes can be prepared to have substantially homogeneous sizes in a selected size range. One effective sizing method involves extruding an aqueous suspension of the liposomes through a series of polycarbonate membranes having a selected uniform pore size; the pore size of the membrane will correspond roughly with the largest sizes of liposomes produced by extrusion through that membrane. See e.g., U.S. Pat. No. 4,737,323, content of which is incorporated herein by reference in its entirety.

The nanoparticles can be in the form of an emulsion. Emulsions are typically heterogeneous systems of one liquid dispersed in another in the form of droplets (Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions may be biphasic systems comprising two immiscible liquid phases intimately mixed and dispersed with each other. In general, emulsions may be of either the water-in-oil (w/o) or the oil-in-water (o/w) variety. When an aqueous phase is finely divided into and dispersed as minute droplets into a bulk oily phase, the resulting composition is called water-in-oil (w/o) emulsion. Alternatively, when an oily phase is finely divided into and dispersed as minute droplets into a bulk aqueous phase, the resulting composition is called an oil-in-water (o/w) emulsion. Emulsions can contain additional components in addition to the dispersed phases, and the conjugate disclosed herein can be present as a solution in either the aqueous phase or the oily phase or itself as a separate phase. Pharmaceutical excipients such as emulsifiers, stabilizers, dyes, and anti-oxidants can also be present in emulsions as needed. Pharmaceutical emulsions can also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous phase provides an o/w/o emulsion.

Emulsifiers can broadly be classified into four categories: synthetic surfactants, naturally occurring emulsifiers, absorption bases, and finely dispersed solids (Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, may be included in the emulsions. See, e.g., Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199. Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: nonionic, anionic, cationic and amphoteric (Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers may be used in the emulsion formulations. Such emulsifiers include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

Non-emulsifying materials may also be included in the emulsion formulations. These include, but are not limited to, fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199). Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

The emulsions may also include preservatives. Commonly used preservatives included in emulsion formulations include methyl paraben, propyl paraben, quaternary ammonium salts, benzalkonium chloride, esters of p-hydroxybenzoic acid, and boric acid. Antioxidants may also be added to the emulsion formulations to prevent deterioration of the formulation. Antioxidants used can be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

Exemplary surfactants for inclusion in the nanoparticles disclosed herein include but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (SO750), decaglycerol decaoleate (DAO750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions can, however, be prepared without the use of cosurfactants and alcohol-free self-emulsifying microemulsion systems are known in the art. The aqueous phase can typically be, but is not limited to, water, an aqueous solution of the drug, glycerol, PEG300, PEG400, polyglycerols, propylene glycols, and derivatives of ethylene glycol. The oil phase can include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Additional active ingredients may also be utilized in the methods and pharmaceutical compositions described herein. In some embodiments, the additional active ingredient is a killer-cell immunoglobulin-like receptors (KIR) inhibitor, T cell immunoreceptor with Ig and ITIM domains (TIGIT) inhibitor, leukocyte-associated immunoglobulin-like receptor 1 (LAIR1) inhibitor, CD160 inhibitor, 2B4 inhibitor, transforming growth factor receptor (TGFR) beta inhibitor, or a combination thereof. In other embodiments, the additional active ingredient is a KIR inhibitor. In further embodiments, the additional active ingredient is a TIGIT inhibitor. In yet other embodiments, the additional active ingredient is a LAIR1 inhibitor. In still further embodiments, the additional active ingredient is a CD160 inhibitor. In other embodiments, the additional active ingredient is a 2B4 inhibitor. In further embodiments, the additional active ingredient is a TGFR beta inhibitor. Other additional active ingredients include, without limitation, toll-like receptor (TLR) agonists, lymphocyte-specific protein tyrosine kinase (LCK) activators, natural killer (NK) cell activators, or granulocyte-macrophage colony-stimulating factor (GM-CSF). These active ingredients may be formulated with a compound described herein, such as the compound of formula (VIII), e.g., Compound 1, or Compound A or A-1, or pharmaceutically acceptable salts thereof, or may be separately administered to the patient as determined by one skilled in the art. These active ingredients also may be formulated with a compound described herein, such as the compound of formula (VIII), e.g., Compound 1, or Compound A or A-1, or pharmaceutically acceptable salts thereof, or may be separately administered to the patient as determined by one skilled in the art.

Thus, in some embodiments, the disclosure provides pharmaceutical compositions comprising a compound described herein, such as the compound of formula (VIII), e.g., Compound 1, Compound A or A-1, or a pharmaceutically acceptable salt thereof, and a pharmaceutically acceptable excipient. In other embodiments, the disclosure provides pharmaceutical compositions comprising nanoparticles comprising a compound described herein, such as a compound of formula (VIII), or a pharmaceutically acceptable salt thereof, and a pharmaceutically acceptable excipient. In further embodiments, the disclosure provides pharmaceutical compositions comprising Compound 1, or a pharmaceutically acceptable salt thereof, and a pharmaceutically acceptable excipient. In other embodiments, the disclosure provides pharmaceutical compositions comprising Compound A or A-1, or a pharmaceutically acceptable salt thereof, and a pharmaceutically acceptable excipient.

The pharmaceutical formulations described herein may be administered by any suitable means including, without limitation, oral, rectal, nasal, parenteral (i.e., subcutaneous, intradermal, intramuscular, intraperitoneal, intravenous, intraarticular, intramedullar), intraperitoneal, transmucosal, transdermal, or topical (i.e., dermal, buccal, sublingual, intraocular). The pharmaceutical formulations are tailored to the particular administration route. Thus, in some embodiments, the pharmaceutical formulations are in the form of tablets, capsules (hard and soft gelatin capsules), sachets, dragees, powders, granules, lozenges, powders for reconstitution, liquid preparations (solutions, emulsions, suspensions, or syrups), patches, inhalants, or suppositories.

The pharmaceutically acceptable excipients are selected based on the mode of administration and may include inert and/or active components. In some embodiments, the pharmaceutically acceptable excipient is sterile, non-toxic, and/or biologically suitable for administration to a subject, i.e., buffered to an appropriate pH and isotonicity. In other embodiments, the pharmaceutically acceptable excipients include diluents (such as inert), carrier, adjuvant, fillers, disintegrants, binders, lubricants, sweeteners, flavors, colors, or preservatives.

Additional embodiments and features of the present disclosure will be apparent to one of ordinary skill in art based upon description provided herein. The embodiments herein provide various features and advantageous details thereof in the description. Descriptions of well-known/conventional methods and techniques are omitted so as to not unnecessarily obscure the embodiments herein. Further, the disclosure herein provides for information and data in the following paragraphs illustrating the above described embodiments, and in order to illustrate the embodiments of the present disclosure certain aspects have been employed. The information and data provided herein for such illustration are intended merely to facilitate an understanding of ways in which the embodiments herein may be practiced and to further enable those of skill in the art to practice the embodiments herein. Accordingly, the following information and examples should not be construed as limiting the scope of the embodiments herein.

### EXAMPLES

### Example 1: Compound 1 Induces ‘Viral Mimicry’ in Cancer Cells Via CMTR2

Following antigen binding, the B-cell receptor (BCR) triggers signaling pathways, which regulates, for instance, the transcription of genes associated with B-cell activation. BCR is internalized and either degraded or trafficked to an intracellular compartment, where complexes containing the antigen bound to the BCR are formed. These complexes are transported to the cell surface, where they are recognized by the T-cell receptor (TCR) of T-helper cells, leading to T-cell activation, by triggering other signalling pathways. The activated T cell in-turn provides ‘help’ to the B cell, leading to full B-cell activation.

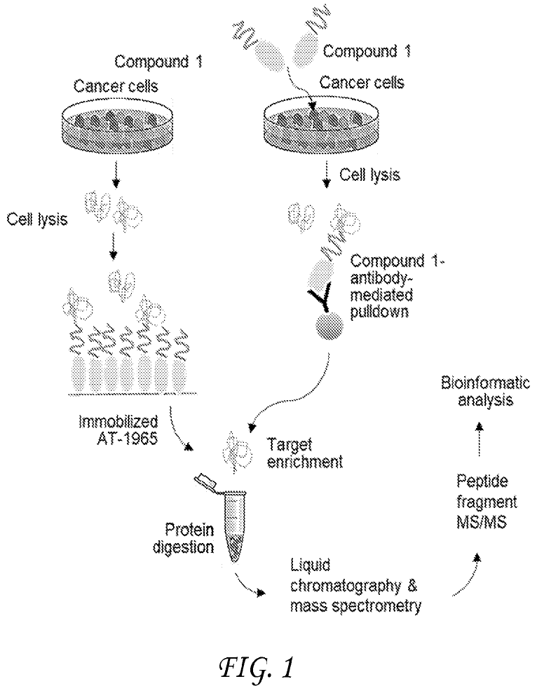

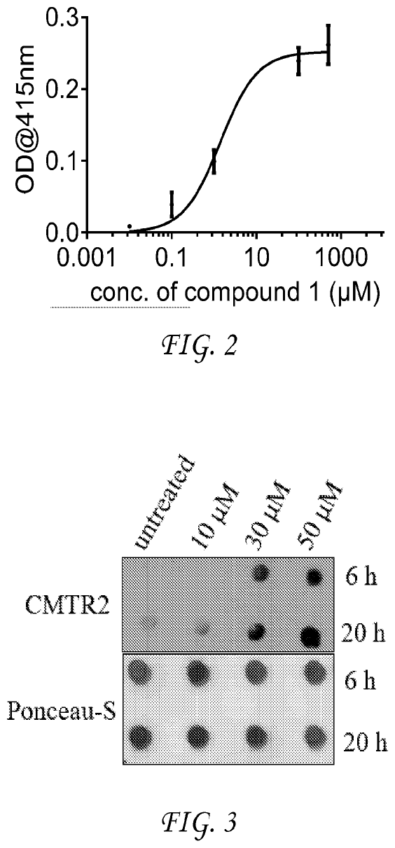

Compound 1 binds to CMTR2, a novel target. Cap-specific mRNA (nucleoside-2′-O-)-methyltransferase 2 (CMTR2) is an enzyme that methylates at the 2′-O position of the second transcribed nucleotide of mRNAs. As suggested by proteomics data (FIG. 1), the Compound 1 binds to CMTR2 from two distinct approaches—(i) anti-cholesterol immunoprecipitations (IP) and (ii) chemical pulldown, where Compound 1 was immobilized on beads, and cell lysate was added to trap binders. This was confirmed by ELISA (FIG. 2). Accordingly, the 4T1 cell lysate demonstrated dose-dependent upregulation of CMTR2 upon treatment with Compound 1.

Further, docking of Compound 1 with CMTR2 reveals that Compound 1 might be binding near the active site thereby potentially inhibiting its activity.

Thus, the present disclosure relates to methods of inhibiting CMTR2. The inhibition is caused by inhibitors including but not limited to compounds of formula (VIII) of the present disclosure. In some aspects, the compound of formula (VIII) is Compound 1. Accordingly, with inhibition of CMTR2, the compound(s) is able to treat cancer as well.

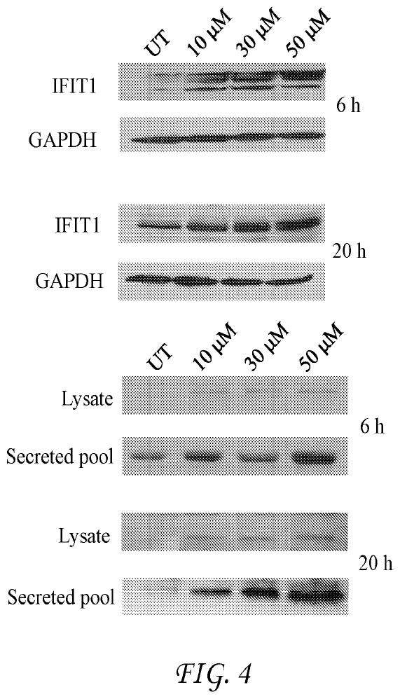

FIG. 3 shows that upon treatment with Compound 1, 4T1 cells upregulate CMTR2 most likely due to a positive feedback loop to compensate for inhibition of CMTR2. Further experiments (FIG. 4) were carried out to suggest that blocking CMTR2 function results in increase of the unmethylated RNA pool which is recognized by Mda5 as viral-like. Alternatively, the unmethylated mRNA could also be recognized by toll-like receptors (TLR) like TLR3. Both of these would trigger many interferon regulatory factors (IRFs) leading to a type-I interferon antiviral response. The interferons can induce interferon stimulated genes (ISGs) such as interferon induced tetratricopeptide repeat protein 1 (IFIT1), which binds to unmethylated mRNA with high affinity. Furthermore, the immune system would be activated by the interferons. Additionally, the unmethylated mRNA could be secreted out from the tumor cells resulting in B-cell activation.

Further, iBlot assay with IFIT1 reveals high levels in both the intracellular lysate and secreted in supernatant (FIG. 4).

Accordingly, the present disclosure relates to methods of treating cancer with a molecule or biologic, such as those including but not limited to Compound 1 that generates unmethylated RNA. The generation of unmethylated RNA is a result of inhibition of CMTR2, and activates B cells, which results in presentation of RNA to BCR.

### Example 2: Viral RNA is then Packaged and Presented to B Cells

Compound 1 aggregates stably interact with the termini of single and double stranded nucleic acids and can lead to the formation of a variety of complex topologies that can be potential B cell activating antigens. A feature is that apart from Compound 1 and nucleic acid strands, no other tertiary biomolecular players are necessary for the formation of these complexes.

To demonstrate this, a short 9 base-pair long single stranded RNA (ssRNA) was simulated in water along with a single Compound 1 molecule. Compound 1 interacts with the termini of the RNA strand and forms transient complexes that associate/dissociate multiple within 200 ns. When the same ssRNA is simulated with five Compound 1 molecules in solution, with the initial configuration such that the Compound 1 molecules are located apart from each other in a sufficiently large box, Compound 1 rapidly aggregates and then interacts with the termini of the ssRNA to form stable complexes, unlike transient ssRNA-single Compound 1 complexes.

Since Compound 1 aggregates formed stable complexes with the termini of short single stranded RNA, simulations were performed to assess whether the Compound 1 aggregates could bind to both termini simultaneously and form RNA loops that resemble double stranded RNA (dsRNA). In the initial configuration, a 14 base pair long RNA strand was simulated in water with a single Compound 1 molecule uniformly distributed in the box. Similar to previous simulations, Compound 1 quickly aggregated, and over the course of the simulation formed a dsRNA-like loop by binding to both termini and bending the strand in the middle.

Compound 1 aggregates also form stable complexes with dsRNA by binding at the termini, similar to DNA and ssRNA. Further, two such Compound 1-dsRNA complexes can rapidly aggregate end-to-end via Compound 1 mediated hydrophobic interactions at the termini to form longer super-complexes. This multimerization could proceed indefinitely, and the resulting super-complexes could probably be antigenic in nature.

Similar to the formation of Compound 1-dsRNA super-complexes, it was shown that a dsDNA-Compound 1 complex and a dsRNA-Compound 1 complex (formed in previous simulations) can rapidly aggregate end-to-end via Compound 1 mediated interactions at the termini and form hybrid DNA-RNA-Compound 1 super-complexes.

All atomistic simulations of Compound 1 subunit with DNA fragment showed permissible phosphate clamps (probability depends on choice of DNA/RNA fragment for simulation) like bonding along the DNA base pairs by accepting hydrogen bonds from two amine ligands. NMR studies carried out on Compound 1 with DNA and RNA bases inferred direct binding of Compound 1 to DNA or RNA as unlikely. This was further supported by molecular dynamics simulations where Compound 1 subunit did not show major/minor grove intercalation with the DNA fragment that is typical of the classical Pt-chemotherapies. From all atom classical molecular dynamics, non-covalent interaction of Compound 1 subunit with the nucleic acid was observed. (Komeda et al., JACS, 2006). A potential implication of the phosphate clamp formation dynamics (as seen in simulation studies) is that methylation of DNA can be perturbed, which could be responsible for the enrichment of dsRNA that was observed in Compound 1 treated samples.

Thus, the present disclosure provides compound(s) such as Compound 1 that interacts with the termini of single and double stranded nucleic acids and lead to formation of a variety of complexes that are B cell activating antigens. Accordingly, the present disclosure relates to methods of treating cancer by activating the B cell mediated immune response, orchestrated by interaction of the compound(s) such as Compound 1 with nucleic acids.

### Example 3: Analysis of Viral RNA and Activation of B Cells

Treatment of cancer cells with Compound 1 results in viral mimicry, and the resultant activation of B cells.

FIG. 5 shows the differential mRNA expression profile of interferon pathway in 4T1 breast cancer. RT-q-PCR was performed with RNA extracted from 4T1 cells treated with 30 μM of Compound 1 or vehicle for 24 h. TLRs, IRF7 and IFN genes showed a robust increase in expression upon Compound 1 treatment. The result is representative of n=3.

In summary, 4T1 cells are treated with Compound 1 at varying concentrations of 10, 20, or 30 μM for 24 hours. The cells are then washed and B-cells added in fresh media. After 24 hours, the B-cells are isolated to determine gene expression by RT-qPCR.

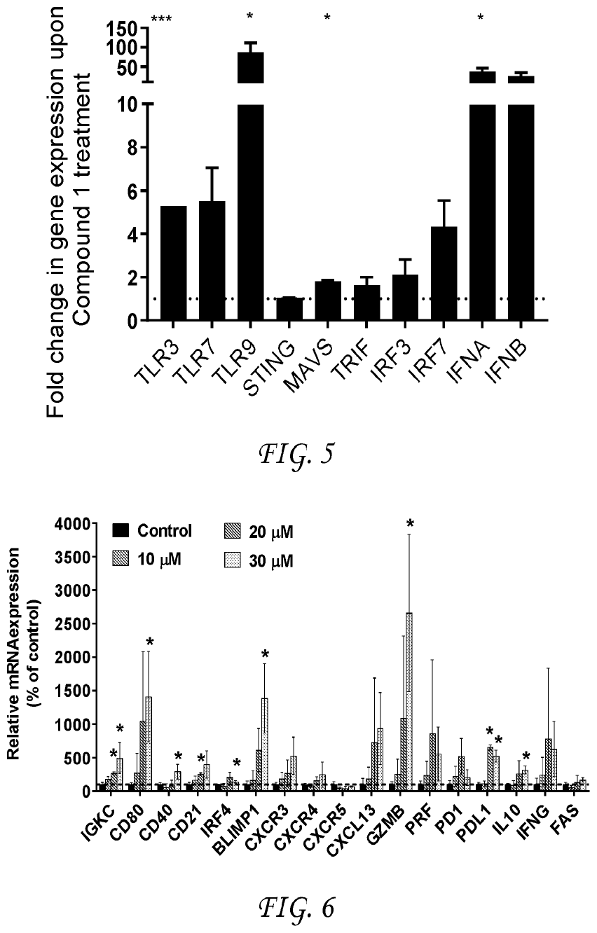

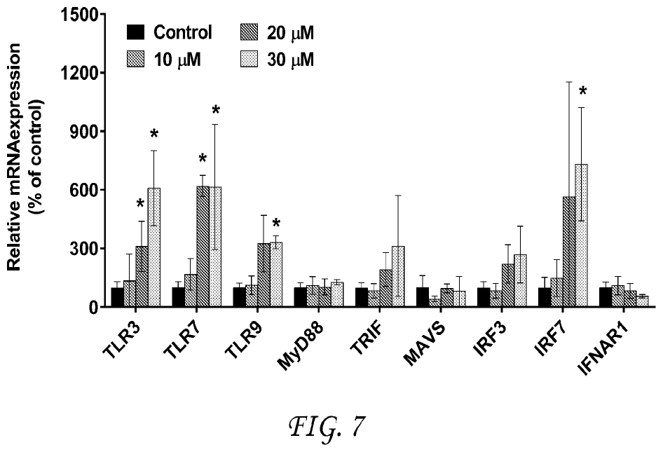

Accordingly, FIGS. 6 and 7 show fold change in transcript levels of B cells after 24 h co-culture with Compound 1 treated 4T1 cells than with non-treated 4T1 cells. Gene expression was normalized to GAPDH and the data is represented as a mean of three-independent experiments (n=3).

Thus, the present disclosure relates to management of cancer by upregulation of B cell mediated immune response caused by B cells activation and differentiation upregulated by compounds such as Compound 1.

### Example 4: Viral Mimicry, B Cells Activation and Tumor Regression In Vivo

Compound 1 regresses breast cancer in vivo with B cell activation.

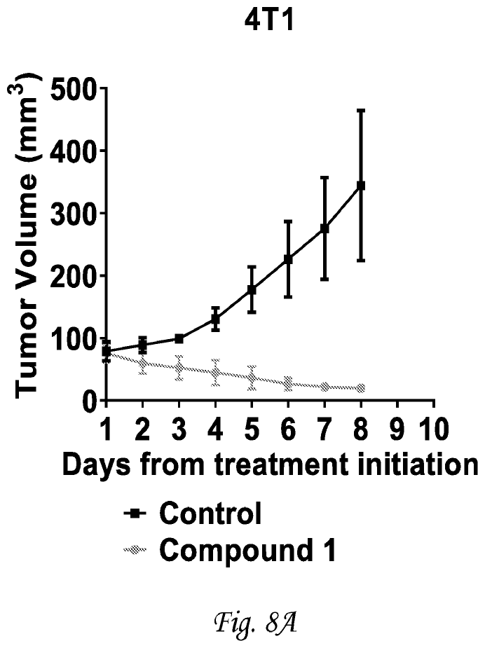

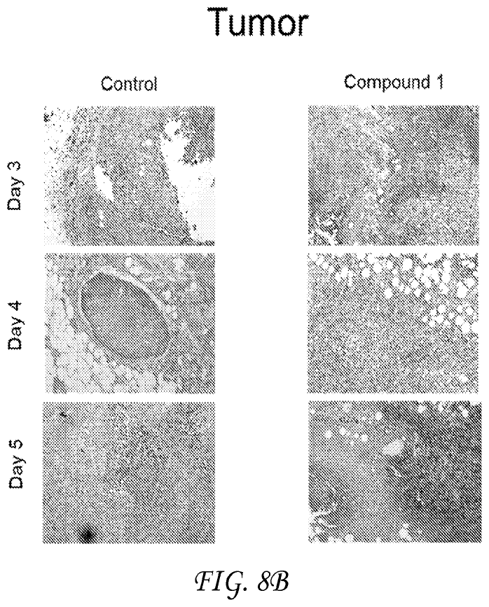

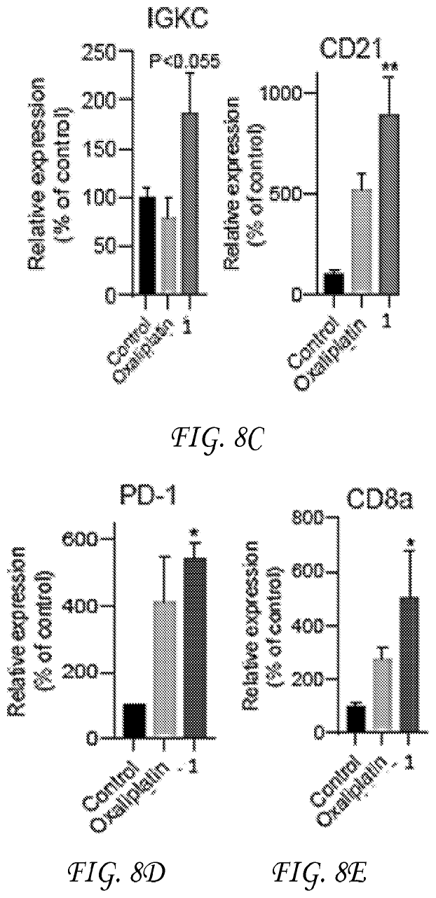

When Compound 1 (cumulative dose of 176 mg/kg per cycle) was administered in 2 cycles, as seen in FIG. 8A, tumor regression is observed of established 4T1 tumors in immunocompetent syngeneic BALB/c mice.

As seen from FIGS. 8B to 8E, an RTPCR analysis of transcripts of biomarkers, which have been associated as a prognostic marker for long term survival in breast cancer patients, reveals an Compound 1-induced increase in IGKC and CD21, which are B cell biomarkers. There was also an increase in CD8a cells.

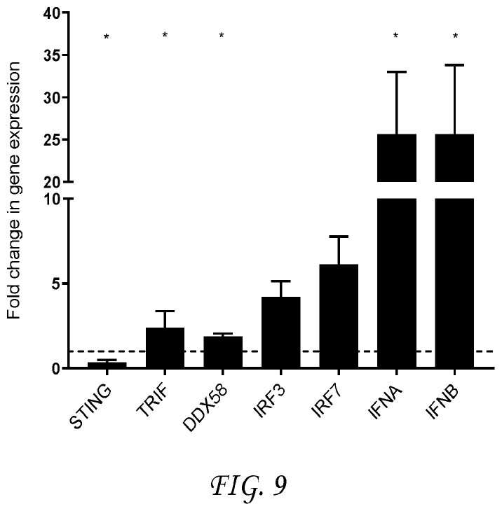

Further, the graph in FIG. 9 shows the gene expression changes in 4T1 tumors that were regressed by 20% on day three following Compound 1 treatment. An increased expression is observed for IRF3 and IRF7. IFN genes also showed a robust increase in expression upon Compound 1 treatment consistent with in vitro observations.

Immunohistological analysis of tumor sections for infiltrating immune cells also was performed. The data illustrated that 50% regressed tumors following Compound 1 treatment showed increased IGKC levels, and increased infiltration of memory B cells (CD20+) and plasma B cells (CD138). Further, the number of CD4+ and CD8+ cells, CD20+ cells (memory B cells), and CD138+ cells (plasma B cell markers) showed a marked increase at day 40. A significant increase in IGKC levels were seen at 40 days in comparison with samples extracted at 10 days. An increased expression is observed for BLIMP1, IRF4, IRF3 was observed. IRF3-dependent type I interferon response in B cells regulates nucleotide-mediated antibody production.

Further, immunofluorescence images of B cells isolated from a Compound 1-treated mouse (with and without tumor) showed the colocalization of HMGB1, a nucleotide sequence-binding protein on the membrane, and the translocation of NFKB to the nucleus.

Accordingly, the present disclosure provides methods of treating/managing cancer by activation of B cells. The activation is caused by compounds including but not limited to the compound of formula (VIII) of the present disclosure (such as Compound 1). In addition, the present disclosure also relates to methods of activating T cells via modulation of cap methyltransferases.

### Example 5: Compound 1-Induced Immune Memory and Role of B Cells In Vivo

Compound 1 Induces Immune Memory.

In Experimental Group 1, the female balb/c mice were injected with 4T1 breast cancer cells. Treatment with Compound 1 was started after the tumors reached ˜80 mm³ in volume. Control group 2 was treated with Compound 1 in parallel but had no tumor to start with. Control group 2 (without tumor) was treated with saline. Twenty-five days post-regression of tumor in experimental group 1, the animals were reinjected with 4T1 cells. In parallel, a similar number of cancer cells were injected in control groups 1 and 2. No additional treatment was administered. All the animals were monitored subsequently for tumor growth.

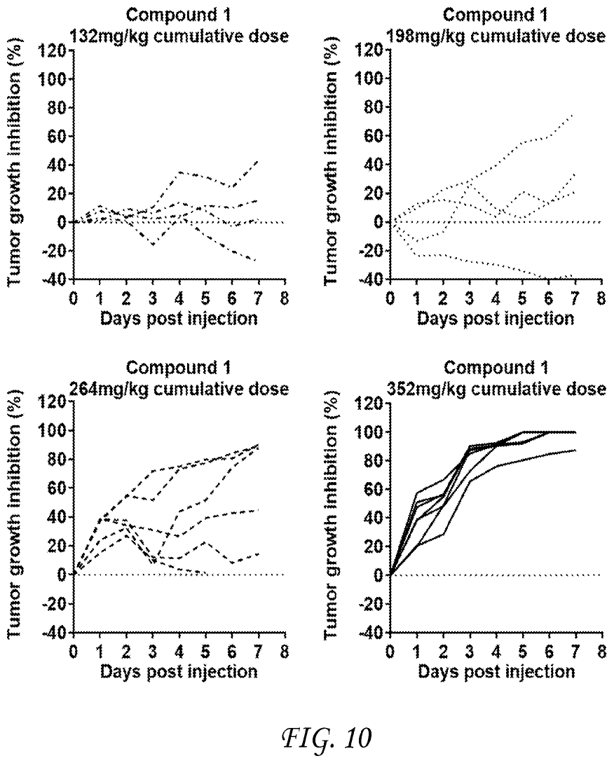

Further, FIG. 10 also shows dose-dependent antitumor effect with Compound 1. 4T1 breast cancer-bearing mice were injected with Compound 1. Individual tumor growth inhibition seen in mice in groups treated with increasing doses of Compound 1. A clear dose-dependent effect is observed.

Thus, the present disclosure provides the use of the compound of formula (VIII) of the present disclosure (such as Compound 1) for regression/treatment/management of cancer.

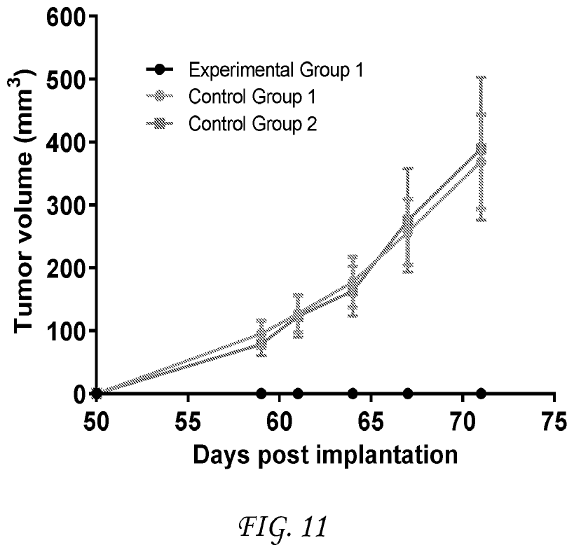

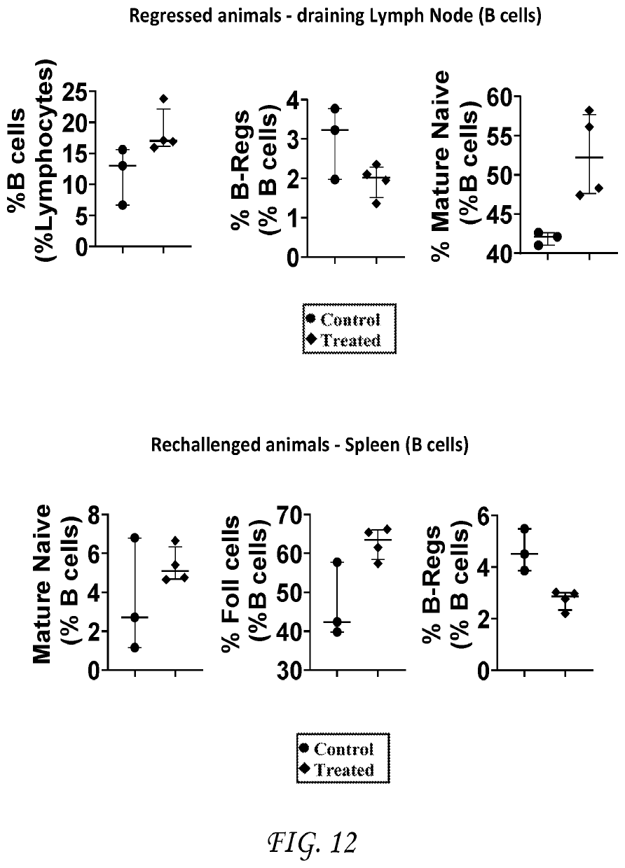

Further, the graph in FIG. 11 shows the absence of any tumor growth in the experimental group post rechallenge with cancer cells, indicating immune memory. In contrast, tumor growth was seen in both control groups, showing that Compound 1-induced immune memory is specific.

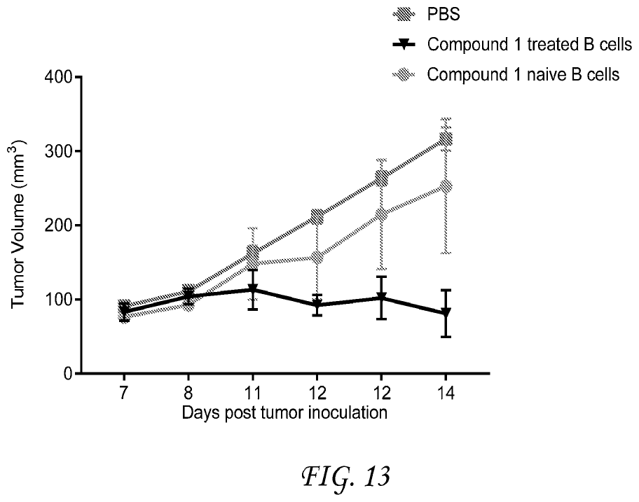

FACS analysis of B cell phenotype in draining lymph node in animals post-regression of tumor following Compound 1-treatment, and in spleen following rechallenge with cancer cells was performed. These data show an increase in B cells with a reduction in Bregs in the Compound 1-treated groups (FIG. 12).

Accordingly, the compounds of formula (VIII) of the present disclosure (such as Compound 1) is not only used for treatment of cancer but also induces immune memory and prevents recurrence of cancer.

Genetic B cell knock-out results in loss of Compound 1 efficacy, whereas adoptive B cell transfer from Compound 1-treated animals confers memory.

Validation of B Cell-Mediated Anticancer Efficacy of Compound 1.

On the other hand, the experimental design for studying the transfer of Compound 1-induced immune memory from one animal to another. Female balb/c mice with established breast cancer were treated with two cycles of Compound 1. Post tumor regression, the animals were sacrificed, and B cells from spleen were isolated and adoptively transferred to a naïve mouse, which was then challenged with 4T1 breast cancer cells. A control animal, without any prior B cell transfer, was similarly injected with cancer cells, and run in parallel. The animals were monitored for cancer growth.

The graph in FIG. 13 further shows that the tumor progression in the B cell adoptive transfer groups was significantly inhibited as compared with the control arm. These results conclusively validate the role of B cells in mediated Compound 1-induced antitumor effects.

Accordingly, the present disclosure provides methods of treating cancer by activating B cells and then adoptively transferring the B cells to exert an anticancer effect. The activation is caused by compounds including but not limited to the compound of formula (VIII) of the present disclosure (such as Compound 1).

### Example 6: Compound 1 can be Combined with Immune Checkpoint Inhibitors

Compound 1 synergizes with PD1 immune checkpoint inhibitor, consistent with T and B cell cooperation in immune rejection.

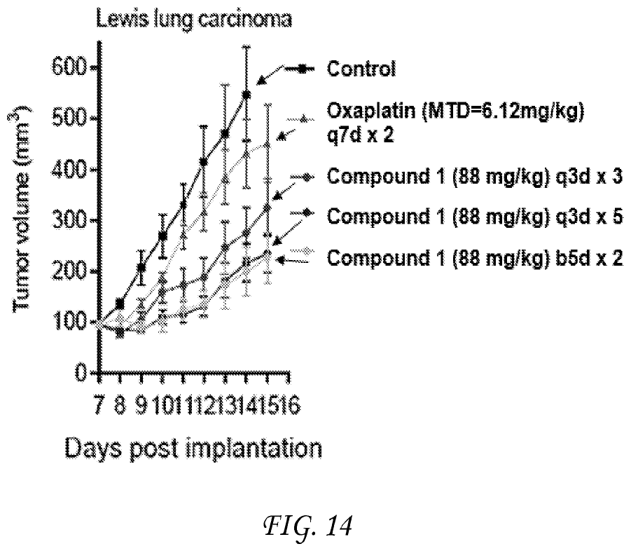

FIG. 14 shows the effect of different dose regimens of Compound 1 on tumor growth in a immunocompetent Lewis lung carcinoma model. Compound 1 treatment resulted in significant tumor growth inhibition as compared with a MTD dose of oxaliplatin. However, no regression was seen in the case of breast cancer.

Analysis of the immuno-transcripts revealed an increase in IGKC signal with Compound 1 treatment, consistent with B cell activation. However, unlike breast cancer, no increase was seen in CD8a levels with Compound 1, which was seen with oxaliplatin. This model was therefore used to test whether the activation of both T cells, using a PD1 inhibitor, and B cells, using Compound 1, results in synergy. In this experiment, four groups of mice models for immunocompetent Lewis lung carcinoma were utilized. The mice were, independently, intravenously treated with control, Compound 1, anti-PD-1, or Compound 1+ anti-PD-1 and tumor growth monitored for at least 28 days post-tumor induction. The following table describes the treatments utilized in the experiment.

| Group | Mice per Group | Compound |
|---|---|---|
| 1 | 6 | Control |
| 2 | 8 | Compound 1 (88 mg/kg per injection) |
| 3 | 8 | anti-PD-1 (2 mg/kg per injection) |
| 4 | 9 | Combo (Compound 1 + anti-PD1) |

The data showed that, while both Compound 1 and PD1 inhibitor resulted in 1 regression and 3 stable disease each, the combination resulted in 4 regressions and 2 stable disease. Thus, in tumors that have TILs, Compound 1 can be used as monotherapy, for example in breast cancer.

Thus, the present disclosure provides the compound of formula (VIII) of the present disclosure (such as Compound 1) for use in regression/treatment/management of cancer.

### Example 7: Formulation and the Stability of Compound 1

Atomistic simulations were used to design formulations in the present disclosure. The simulations show the self-assembly of Compound 1 with lipids into a planar lipid bilayer. A homogenous formulation of Compound 1 and co-lipids in the ratio of 38:62, with ˜50 water molecules per solute molecule, assembles spontaneously into a lipid membrane.

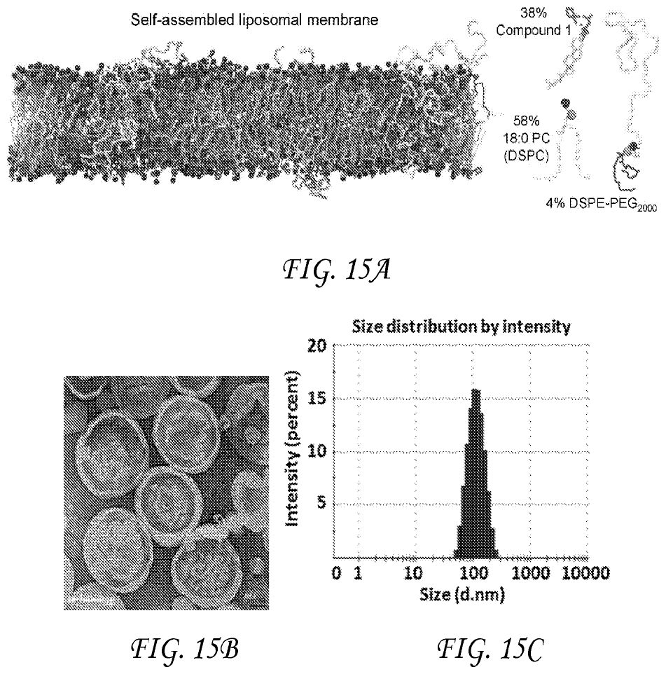

FIGS. 15A-15C show self-assembly of liposomes and Cryo-EM images of the supramolecular liposomes and the narrow distribution of size ˜100 nm as measured by dynamic laser light scattering. 1H NMR spectra of the reaction of a) 5′ GMP and oxaliplatin (New peak at 8.5 ppm is due to Pt-GMP adduct formation); c) Compound 1 subunit and 5′ GMP (no adduct formation observed); b) Compound 1 M and 5′ GMP (no adduct formation observed) showed that unlike oxaliplatin, Compound 1 does not react with DNA nucleotide.

Further, to understand whether Compound 1 also behaves similarly, the distribution of Compound 1 in various compartments of the cell was studied. 4T1 cells treated with 50 μM Compound 1 and oxaliplatin were lysed using NE-PER kit (Thermo Scientific) to separate the cytosolic and nuclear protein fractions. The remaining insoluble proteins were resuspended in RIPA lysis buffer. The leftover genomic DNA (gDNA) pellet was treated with methanol to dissociate any non-covalently bound platinum from the gDNA. All the fractions were assayed by atomic absorption spectroscopy (AAS) to determine their Pt content. Significant differences were observed in the distribution of platinum between the fractions from cells treated with the two drugs. This implies that Compound 1 does not form the canonical Pt-adducts with DNA as known for oxaliplatin and has a differential mechanism of action than the classical Pt-drugs. This is further validated by the equivalency in intact Compound 1 concentration by RPHPLC and platinum concentration by AAS demonstrates that Compound 1 remains intact intracellularly. Data was generated from three independent biological experiments and error represents standard error of mean.

The present disclosure therefore relates to anti-tumor compounds such as Compound 1 and formulations thereof for use in management/treatment of cancer. This treatment is a direct result of interaction of Compound 1 with different components of the immune system, particularly the acquired immune system.

### Example 8: PK Parameters (Non-Compartmental Analysis) of Compound 1

The pharmacokinetic (PK) profile of Compound 1 was evaluated following single intravenous (i.v bolus) dosing in tumor bearing female Balb/c mice. Tumor and plasma samples were collected at various time points, sparse sampling (n=3). Plasma concentrations were determined using LC-MS/MS and later subjected to Phoenix WinNonlin Noncompartmental analysis and PK parameters were estimated.

| Male | | Dose (mg/kg) | 0 | | 44 | | 88 | | | |
|---|---|---|---|---|---|---|---|---|---|---|
| | | Group | G1 | | G2 | | G3 | | | |
| (Bone Marrow) Hematology | | No. of animals | N = 5 | | N = 5 | | N = 5 | | | |
| | Parameter | Unit | Mean | SD | Mean | SD | Mean | SD | | |
| Anemia | Hemoglobin | (g/dL) | 15.0 | 0.51 | 15.84 | 0.6 | 14.92 | 0.20 | | |
| Thrombocytopenia | Platelet | (× 10³ cells/μL) | 1155.8 | 109.32 | 1201.2 | 79 | 1376.2 | 52.1 | | |
| Neutropenia | Neutrophil | (× 10³ cells/μL) | 0.63 | 0.08 | 0.72 | 0.3 | 0.80 | 0.32 | | |
| Female | | Dose (mg/kg) | 0 | | 44 | | 88 | | 176 | |
| | | Group | G1 | | G2 | | G3 | | G4 | |
| Clinical Chemistry | | No. of animals | N = 5 | | N = 5 | | N = 5 | | N = 2 | |
| | Parameter | Unit | Mean | SD | Mean | SD | Mean | SD | Mean | SD |
| Hepatic | AST | (U/L) | 138.00 | 23.57 | 110.60 | 8.65 | 106.00 | 12.6 | 141.50 | 16.26 |
| | ALT | (U/L) | 29.40 | 3.71 | 30.00 | 4.12 | 25.60 | 4.16 | 42.50 | 3.54 |
| | ALP | (U/L) | 118.40 | 23.47 | 107.60 | 14.48 | 133.00 | 11.49 | 209.5 | 74.25 |
| Renal | Creatinine | (mg/dL) | 0.49 | 0.08 | 0.49 | 0.07 | 0.49 | 0.03 | 0.52 | 0.04 |

The estimated AUC levels for Compound 1 in mice increased linearly with dose. PK studies in rats, dogs show nice dose conversion.

Histological evaluation of tissues from mice treated with Compound 1 (198 mg/kg; cumulative dose) was performed, where Compound 1 treatment showed minimal abnormality in tissues. Further, Compound 1 was also tested for safety end point evaluation in rats. In a single dose toxicity study, Sprague Dawley rats of both the genders were subjected to intravenous infusion (30 min) dose of 44, 88 and 176 mg/kg respectively and were observed for clinical signs, mortality, hematology and clinical chemistry. Doses of 44 mg/kg, 88 mg/kg and 176 mg/kg were tested and 176 mg/kg was LTD. The remaining doses were well tolerated without any significant clinical signs and all other parameters were comparable to control group.

### Example 9: Compound 1 Inhibits CMTR2 and Induces Viral Mimicry

In this example, two different proteomics approach were used to identify the target of Compound 1: (i) an anti-drug antibody (binds to cholesterol in Compound 1) and (ii) Compound A-1, where both (i) and (ii) were used to precipitate targets from 4T1 lysates. The 4T1 cell lysate was treated with pegylated Compound 1 for 20 hours. The 4T1 cells lysate was, independently, combined with the proteins of Table A or treated with Compound 1 and then combined with the proteins of Table A. Precipitated proteins were digested into peptides and identified using LC-MS/MS. Table A provides a summary of the proteins identified from both approaches that were enriched in Compound 1 samples above the background. These results illustrate that CMTR2 appeared as the lead putative target of Compound 1 with the only protein with a false discovery rate of less than 1%.

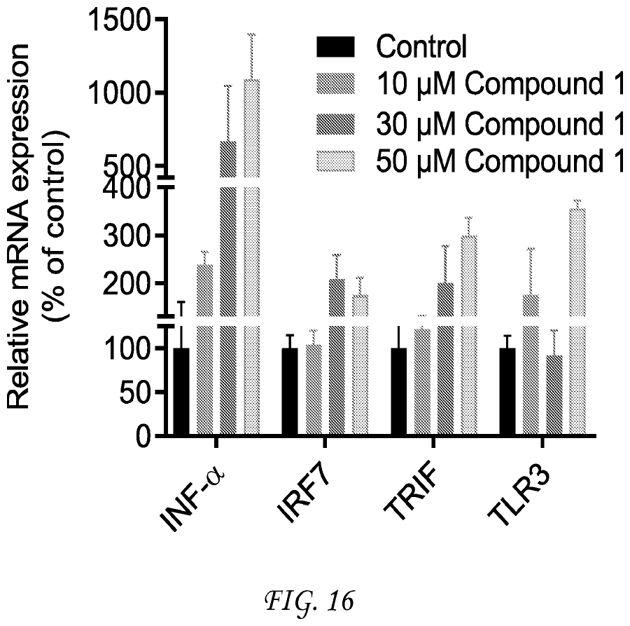

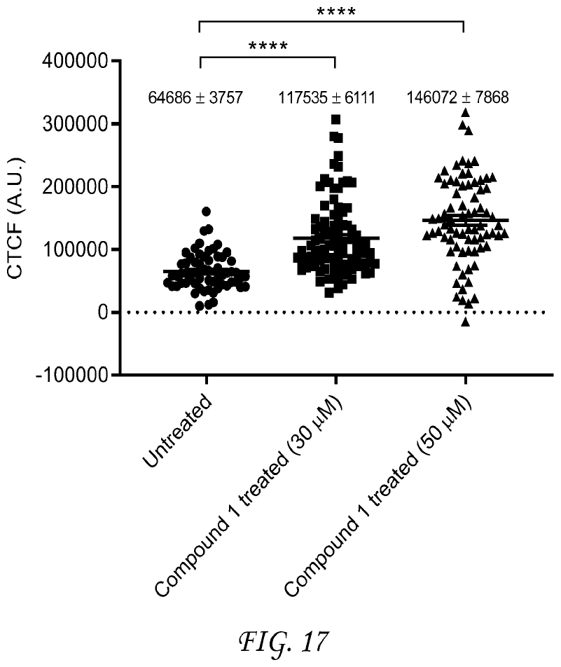

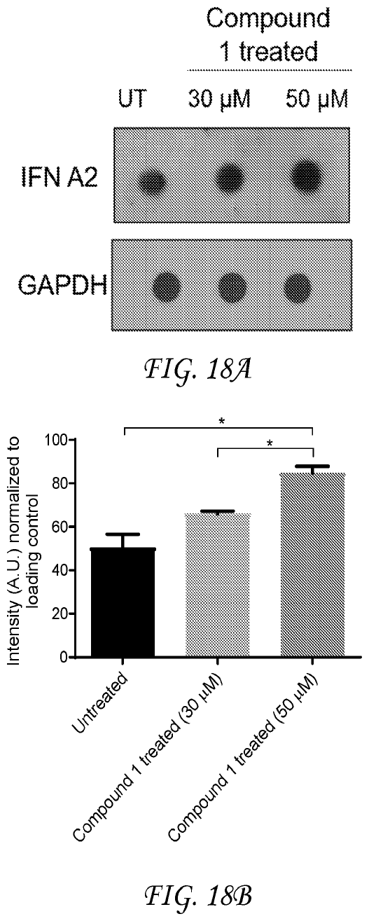

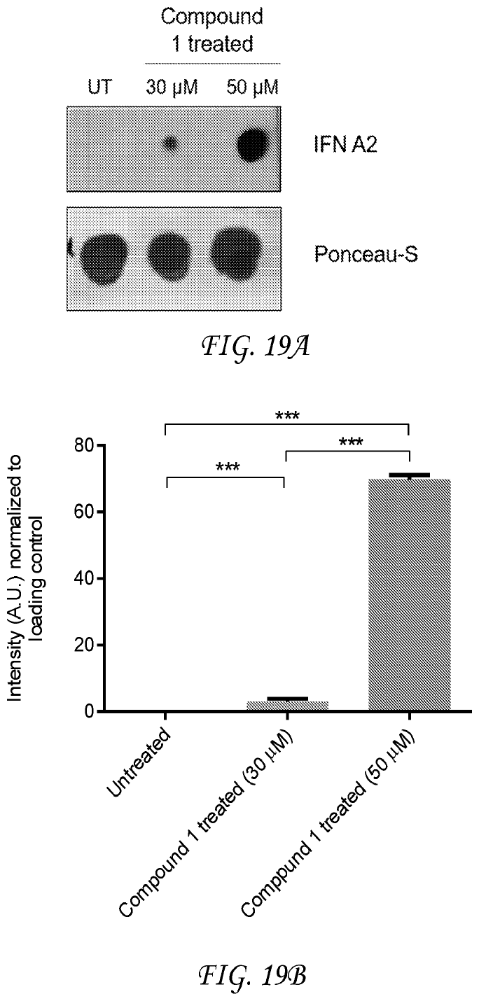

The 4T1 cells that were treated with Compound 1 showed activation of the innate anti-viral response in a dose-dependent manner. See, FIG. 16. The unmethylated mRNA generated due to inhibition of CMTR2 activity leads to recognition by sensors like MDA5 and TLR3 in conjunction with adapters like TRIF. This results in activation of transcription factors such as IRF3 and IRF7 which stimulate type-I interferon production (INF-α). FIG. 17 shows that IRF7, an inducible interferon stimulated transcription factor, showed increased levels in 4T1 cells in a dose-dependent manner. FIGS. 18A-18B showed that INF-α levels were significantly elevated in 4T1 cells treated with Compound 1 in a dose-dependent manner. Similarly, INF-α levels were remarkably high in the secreted pool of 4T1 cells treated with Compound 1, as shown in FIG. 19A-19B.

### Example 10: Compound 1 Activation of B-Cells In Vitro

In this example, experiments were performed using Compound 1 to show its activation of B-cells via a IFIT1-mRNA complex. The complex binds B-cell receptor on the surface of the B-cells which initiates a signalling cascade via the pSYK-PLCy2-NFkB-IRF pathway leading to cytokine and auto-antibodies production.

IFIT1-mRNA complex was isolated and shown to co-localize with IgM (a component of the B-cell receptor) on the surface of B-cells implicating the antigenic potential of the IFIT1-mRNA complex.

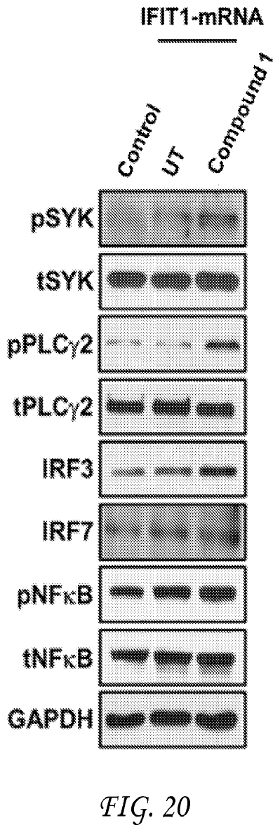

B-cells incubated with isolated IFIT1-mRNA complex from untreated and Compound 1 treated 4T1 cells. FIG. 20 shows the activation of B-cells via the pSYK-PLCy2-NFkB-IRF pathway. 4T1 cells treated with Compound 1 when co-cultured with B-cells showed similar activation of the pathway in dose-dependent and time-dependent manner.

### Example 11: Compound 1 Induces Viral Mimicry In Vivo Thereby Activating B-Cells

Compound 1 was tested in vivo in female Balb/c mice implanted with 4T1 tumors. Specifically, Compound 1 was administered to mice on days 1, 2, 6, and 7 and the tumor growth and body weight of the mice monitored. On day 8, plasma samples were collected.

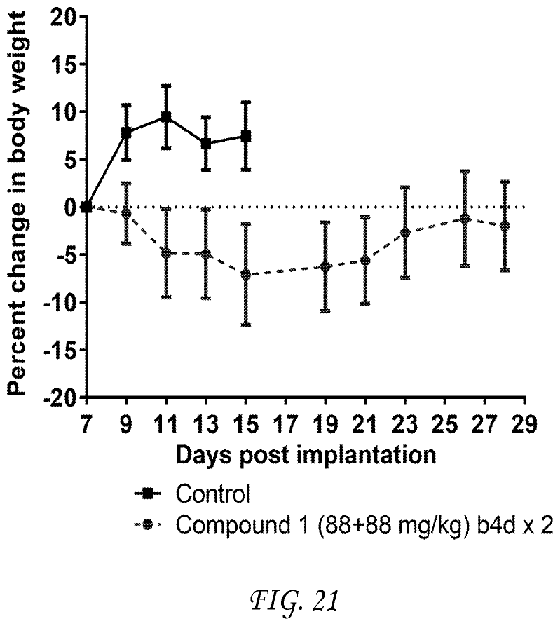

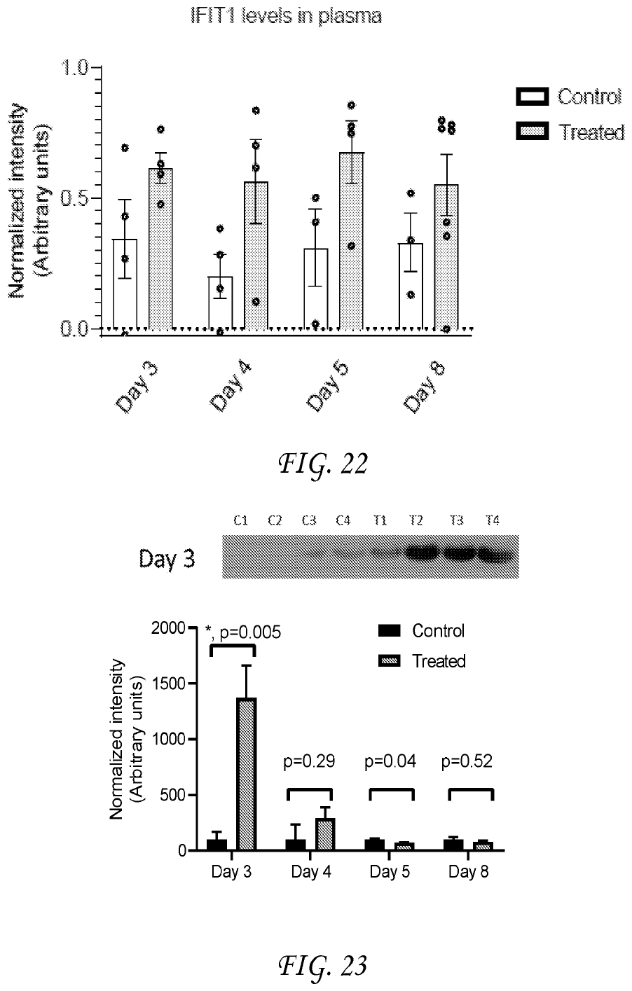

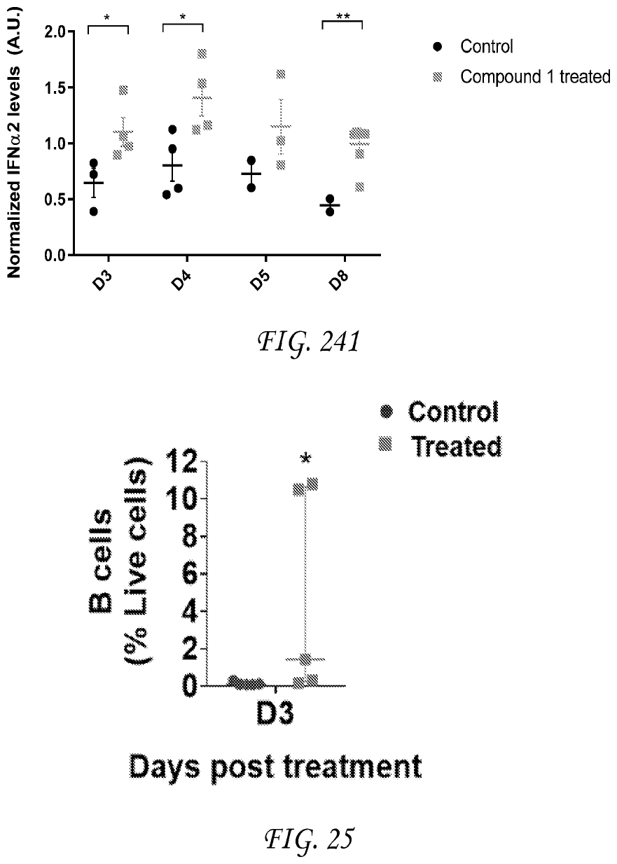

These results showed that Compound 1 ablated 4T1 tumors in vivo in mice without any loss in body weight. See, FIG. 21. These results also showed that IFIT1 levels in plasma were elevated on days 3 to 8 as measured by dot-blot. See, FIG. 22. The circulating IFIT1 was also mRNA bound as shown by western blot analysis of oligo-dT pull downs from plasma. See, FIG. 23. The INF-α levels were also elevated in plasma suggesting that the viral mimicry response observed in vitro was also translated in vivo. See, FIG. 24. IGKC, a marker for B-cells was found to be overexpressed by immunohistochemical analysis of tumor sections on day 3 and in spleen on day 4. Finally, B-cells were found to be infiltrating the tumor upon Compound 1 treatment on day 3 as reflected by measurement of % B-cell by FACS. See, FIG. 25.

### Example 12: Compound 1 Activity is B-Cell Dependent In Vivo

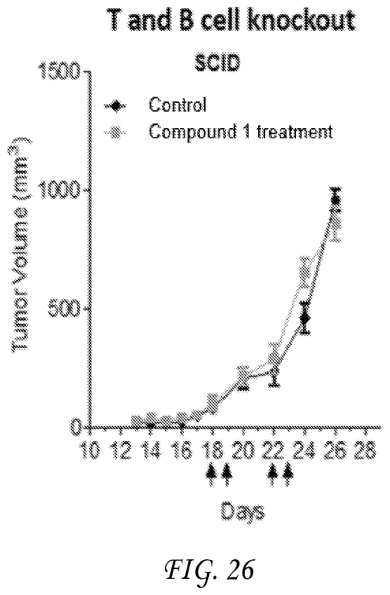

Compound 1 was tested in vivo in female Balb/c mice implanted with 4T1 tumors in 3 groups. In summary, the mice were treated with a saline solution or a 88 mg/kg Compound 1 solution in 2 cycles. Mice were then rechallenged with 4T1 tumors and the tumor growth monitored. The results illustrate that 4T1 tumor ablation does not happen in mice lacking B cells, validating the role of B cell-mediated tumor regression upon treatment with Compound 1. See, FIG. 26. No tumor growth occurs following re-implantation of cancer cells in the animals which had previously undergone tumor regression with Compound 1, consistent with the development of B-cell mediated immune memory. See, FIG. 27.

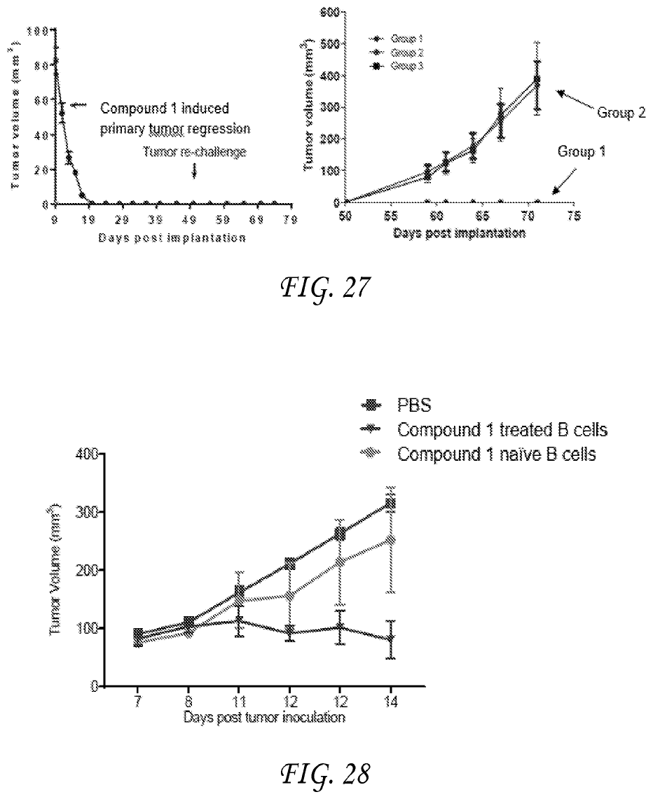

In another study, Compound 1 was administered to mice on days 1, 2, 6, and 7 and the tumor growth and body weight of the mice monitored. On day 9, the spleen were harvested and the B-cells purified. The same B-cells isolated from the mice were then injected into a female Balb/c mouse, 4T1 cells injected subcutaneously in the mouse after 46 hours, and then tumor growth and body weight of the mouse monitored. Upon injection of B-cells harvested from mice in which tumor regressed post Compound 1 treatment were in mice challenged with 4T1 tumor resulted in tumor regression implying the priming of B-cells against the neo-antigens generated from tumor after Compound 1 treatment. See, FIG. 28.

Thus, the present disclosure relates to methods of treating cancer by modulating cap methyltransferase enzyme.

The present disclosure accordingly also provides methods of treating cancer by inhibiting cap methyltransferase using pharmacological or biological inhibitors. One such inhibitor is a compound described herein, such as the compound of formula (VIII) of the present disclosure (Compound 1) or Compound A or A-1.

Further, the present disclosure also provides methods of treating cancer with a molecule or biologic that generates unmethylated RNA. One such molecule or biologic is a compound described herein, such as the compound of formula (VIII) of the present disclosure (such as Compound 1) or Compound A or A-1.

The present disclosure also provide methods of activation of B cells by generation of unmethylated RNA and presenting them to BCR.

The present disclosure further provides methods of treating cancer by activation of B cells and then adoptively transferring the B cells to exert an anticancer effect. One such activator is a compound described herein, such as the compound of formula (VIII) of the present disclosure (Compound 1) or Compound A or A-1.

The present disclosure also provides methods of activating B cells via modulation of cap methyltransferases. One such modulator is a compound described herein, such as the compound of formula (VIII) of the present disclosure (such as Compound 1) or Compound A or A-1.

Further, the present disclosure also provides methods of activating T cells via modulation of cap methyltransferases. One such modulator is a compound described herein, such as the compound of formula (VIII) of the present disclosure (such as Compound 1) or Compound A or A-1.

The present disclosure also relates to use of cap methyltransferase inhibitors for treating cancer. One such cap methyltransferase inhibitor is a compound described herein, such as the compound of formula (VIII) of the present disclosure (such as Compound 1) or Compound A or A-1. Accordingly, the disclosure provides the Compound 1 for inhibition of cap methyltransferase, and in-turn management of cancer. The disclosure also provides Compound A or A-1 for inhibiting cap methyltransferase, and in-turn managing cancer.

Although the disclosure and exemplification has been provided by way of illustrations and experiments for the purpose of clarity and understanding, it is apparent to a person skilled in the art that various changes and modifications can be practiced without departing from the spirit or scope of the disclosure. Accordingly, the foregoing descriptions and experiments should not be construed as limiting the scope of the present disclosure.

The description of the embodiments of the present disclosure reveals the general nature of the embodiments that are readily suitable for modification and/or adaptation for various applications by applying the current knowledge. Such specific embodiments of the disclosure, without departing from the generic concept, and, therefore, such adaptations and modifications should and are intended to be comprehended and considered within the meaning and range of equivalents of the disclosed embodiments.

Any discussion of documents, acts, materials, devices, articles and the like that has been included in this specification is solely for the purpose of providing a context for the present disclosure. It is not to be taken as an admission that any or all of these matters form a part of the prior art base or are common general knowledge in the field relevant to the present disclosure, as it existed anywhere before the priority date of this application.

The contents of all references, patents, and published patent applications cited throughout this application are incorporated herein by reference for all purposes.

1. A method for modulating cap methyltransferase enzyme 2 (CMTR2), comprising administering a compound of formula (VIII) to a patient in need thereof:
Q-linker-lipid (VIII)
wherein:
Q is a platinum containing moiety; and
the linker has at least one linkage to the platinum atom.

2. The method of claim 1, wherein Q is:
(i) 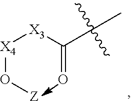
wherein:
X₃ is (CH₂)ₙ, CH₂—NH, or C₄Hs;
X₄ is CO or —CH(CH₃)—;
Z is a platinum containing compound, wherein the platinum forms a part of the ring; and
n is 0, 1, or 2;
(ii) 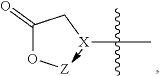
wherein:
X is NH or N(CH₂COO⁻); and
Z is a platinum containing compound, wherein the platinum forms a part of the ring;
(iii) 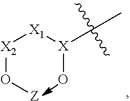
wherein:
X is S⁺, C, S⁺═O, N⁺H, or P═O;
X₁ is CH, CH₂ or CH₂O;
X₂ is C(O); and
Z is a platinum containing compound, wherein the platinum forms a part of the ring;
(vi) 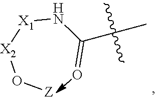
wherein:
X₁ is (CH₂)ₙ;
X₂ is C(O);
Z is a platinum containing compound, wherein the platinum forms a part of the ring; and
n is 0, 1, or 2; or
(v) 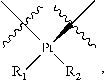
wherein:
R₁ and R₂ are, independently, halogen, alkyl, amino, alkylamino, dialkylamino, hydroxyl, alkoxy, thiol, thioalkyl, O-acyl, or a combination thereof;
(vi) 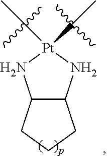
wherein:
p is 0, 1, 2, or 3;
(vii) 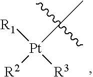
wherein:
R¹, R² and R³ are, independently, halogen, alkyl, amino, alkylamino, dialkylamino, hydroxyl, alkoxy, thiol, thioalkyl, O-acyl, or a combination thereof;
(viii) 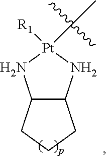
wherein:
R¹ is halogen, alkyl, amino, alkylamino, dialkylamino, hydroxyl, alkoxy, thiol, thioalkyl, O-acyl, or a combination thereof; and
p is 0, 1, 2, or 3;
(ix) 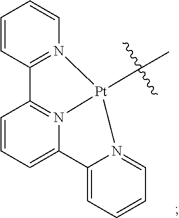
(x) 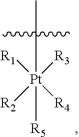
wherein:
R₁, R₂, R₃, R₄ and R₅ are, independently, halogen, alkyl, amino, alkylamino, dialkylamino, hydroxyl, alkoxy, thiol, thioalkyl, O-acyl, or a combination thereof; or
(xi) 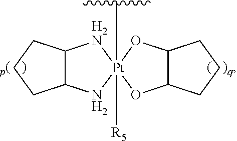
wherein:
p an q are both 2; and
R₅ is OH, OC(O)CH₃, or OC(O)-phenyl.

3. The method of claim 2, wherein Z is:
(a) 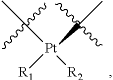
wherein:
R₁ and R₂ are, independently, halogen, alkyl, amino, alkylamino, dialkylamino, hydroxyl, alkoxy, thiol, thioalkyl, O-acyl, or a combination thereof;
(b) 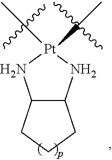
wherein:
p is 0, 1, 2, or 3;
(c) 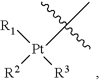
wherein:
R¹, R² and R³ are, independently, halogen, alkyl, amino, alkylamino, dialkylamino, hydroxyl, alkoxy, thiol, thioalkyl, O-acyl, -linker-lipid, or a combination thereof,
(d) 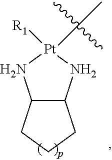
wherein:
R₁ is halogen, alkyl, amino, alkylamino, dialkylamino, hydroxyl, alkoxy, thiol, thioalkyl, O-acyl, or a combination thereof; and
p is 0, 1, 2, or 3;
(e) 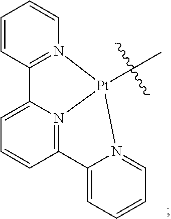
(f) 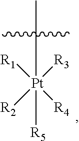
wherein:
R₁, R₂, R₃, R₄ and R₅ are, independently, halogen, alkyl, amino, alkylamino, dialkylamino, hydroxyl, alkoxy, thiol, thioalkyl, O-acyl, -linker-lipid, or a combination thereof,
(g) 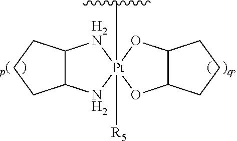
wherein:
p and q are, independently, 0, 1, 2, or 3; and
R₅ is OH, OC(O)CH₃, or OC(O)-phenyl; or
(h) 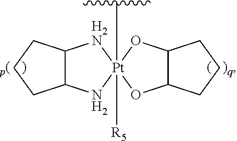
wherein:
p and q are both 2; and
R₅ is OH, OC(O)CH₃, or OC(O)-phenyl.

4. The method of claim 1, wherein the lipid is a fat, wax, sterol, steroid, bile acid, fat-soluble vitamin (such as A, D, E, and K), glycerolipid (such as a monoglyceride, diglyceride, or triglyceride), phospholipid, glycolipid, sulpholipid, aminolipid, chromolipid (lipochrome), glycerophospholipid, sphingolipid, prenol lipid, saccharolipid, polyketide, fatty acid, or fatty alcohol, or a combination thereof.

5. (canceled)

6. The method of claim 1, wherein the lipid is cholesterol.

7. The method of claim 1, wherein the linker is:
(a) —X—CH₂—X₂—X₁—, wherein:
X is NH;
X₁ is C(O)O, C(O)NH, O(CH₂)O, NH, or O;
X₂ is (CH₂)ₙ or C(O); and
n is 0, 1, 2, 3, 4, or 5;
(b) —(CH₂)ₙO—, —(CH₂)ₙNHC(O)O—, —(CH₂)ₙOC(O)NH—, —(CH₂)ₙC(O)NH(CH₂)ₘO—, —(CH₂)ₙO(CH₂)ₘO—, —(CH₂)ₙC(O)—, —(CH₂)ₙNHC(O)(CH₂)ₘO—, or —(CH₂)ₙC(O)O—; and n and m are independently 0, 1, 2, 3, 4, or 5;
(c) —X₃—X₄X₅—X₆—, wherein:
X₃ is CH, CH₂, or O; and
X₄, X₅ and X₆ are independently same or different and are —CH₂O— or O;
(d) a bond, —O—, NHCH₂CH₂NHC(O)—, —NHCH₂CH₂NHC(O)O—, —NHCH₂CH₂—, —NHCH₂CH₂O—, —NHCH₂C(O)—, —NHCH₂C(O)O—, —NHCH₂C(O)OCH₂CH₂CH₂—, —NHCH₂C(O)OCH₂CH₂CH₂O—, —NHCH₂C(O)NH—, —CH₂CH₂—, —CH₂CH₂O—, —CH₂CH₂NHC(O)—, —CH₂CH₂NHC(O)O—, —CH₂CH₂O—, —CH₂C(O)NHCH₂CH₂—, —CH₂C(O)NHCH₂CH₂O—, —CH₂CH₂OCH₂CH₂—, —CH₂CH₂OCH₂CH₂O—, —CH₂C(O)—, —CH₂C(O)O—, CH₂CH₂CH₂—, —CH₂CH₂CH₂O—, ═CHCH═CH₂—, ═CH—CH═CHCH₂O—, —CH═CHCH₂—, —CH═CHCH₂O—, —OCH₂CH₂O—, —CH₂—, —CH₂O—, —NHC(O)CH₂—, —NHC(O)CH₂O—, —C(O)CH₂—, —C(O)CH₂O—, —OC(O)CH₂—, —OC(O)CH₂O—, —C(O)CH₂CH₂C(O)NHCH₂CH₂—, —OC(O)CH₂CH₂C(O)NHCH₂CH₂—, —C(O)CH₂CH₂C(O)NHCH₂CH₂O—, —OC(O)CH₂CH₂C(O)NHCH₂CH₂O—, —C(O)CH₂CH₂C(O)NHCH₂CH₂NHC(O)—, —OC(O)CH₂CH₂C(O)NHCH₂CH₂NHC(O)—, —C(O)CH₂CH₂C(O)NHCH₂CH₂NHC(O)O—, —OC(O)CH₂CH₂C(O)NHCH₂CH₂NHC(O)O—, or a combination thereof; or
(e) a combination of (a)-(c).

8. The method of claim 1, wherein the compound is:
(i) a compound of Formula (I): 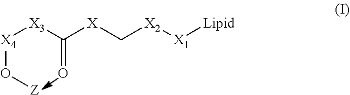
wherein:
X is NH;
X₁ is —C(O)O—, —C(O)NH—, —O(CH₂)ₙO—, NH, or O;
X₂ is (CH₂)ₙ or C(O);
X₃ is (CH₂)ₙ, —CH₂NH—, or C₄H₈;
X₄ is C(O) or —CH(CH₃)—;
Z is a platinum containing compound, wherein the platinum forms a part of Formula I ring; and
n is 0, 1, or 2;
(ii) a compound Formula (II): 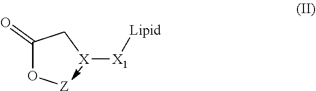
wherein:
X is NH or NCH₂COO⁻;
X₁ is —(CH₂)ₙO—, —(CH₂)ₙNHC(O)O—, —(CH₂)ₙC(O)NH(CH₂)ₙO—, —(CH₂)ₙO(CH₂)ₙO—, —(CH₂)ₙC(O)—, —(CH₂)ₙNHC(O)(CH₂)ₙO—, or —(CH₂)ₙC(O)O—;
Z is platinum containing compound, wherein the platinum forms a part of Formula II ring; and
n is 0, 1, or 2;
(iii) a compound of Formula (III): 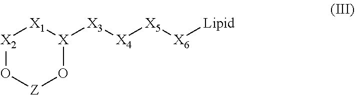
wherein:
X is S⁺, C, S⁺═O, N⁺H, or P═O;
X₁ is CH, CH₂ or CH₂O;
X₂ is C(O);
X₃ is CH, CH₂ or O;
X₄, X₅, and X₆ are, independently, CH₂O or O;
Z is platinum containing compound, wherein the platinum forms a part of Formula III ring; or
(iv) a compound of Formula (IV): 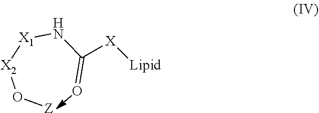
wherein:
X is CH₂O;
X₁ is (CH₂)ₙ;
X₂ is C(O);
Z is platinum containing compound, wherein the platinum forms a part of Formula IV ring; and
n is 0, 1, or 2.

9. The method of claim 1, wherein the compound of formula (VIII) is Compound 1:

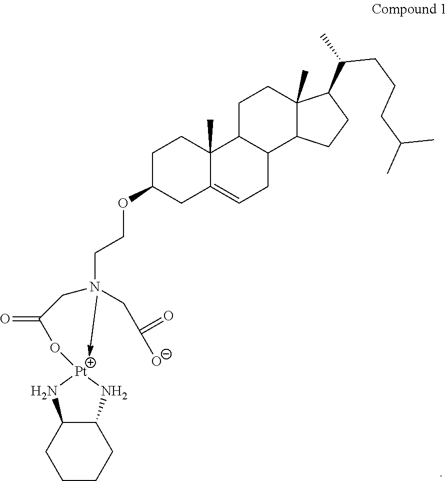

10. The method of claim 1, wherein the compound is formulated in a composition.

11. The method of claim 9, wherein the composition comprises nanoparticles of the compound of formula (VIII).

12. The method of claim 9, wherein the composition comprises one or more lipids.

13. The method of claim 12, wherein the lipid is 1,2-distearoyl-sn-glycero-3-phosphonoethanolamine-N-[methoxy(polyethyleneglycol)-2000](ammonium salt) or L-α-phospatidylcholine, hydrogenated (soy).

14. (canceled)

15. The method of claim 1, wherein said modulating is inhibiting.

16. The method of claim 1, comprising inducing an immune response in the patient.

17. The method of claim 15, wherein inhibiting CMTR2 activates T cells or B cells.

18. (canceled)

19. The method of claim 1, wherein the patient has cancer.

20. The method of claim 19, wherein the cancer is caused by B-cell depletion, T-cell depletion, or a combination thereof.

21. (canceled)

22. The method of claim 19, wherein the cancer is lymphoma, stomach cancer, breast cancer, bladder cancer, or cervical epithelial cancer.

23. The method of claim 1, wherein the subject has common variable immune deficiency (CVID), optionally associated with defective humoral immunity.

24-46. (canceled)

47. A compound of formula (A): 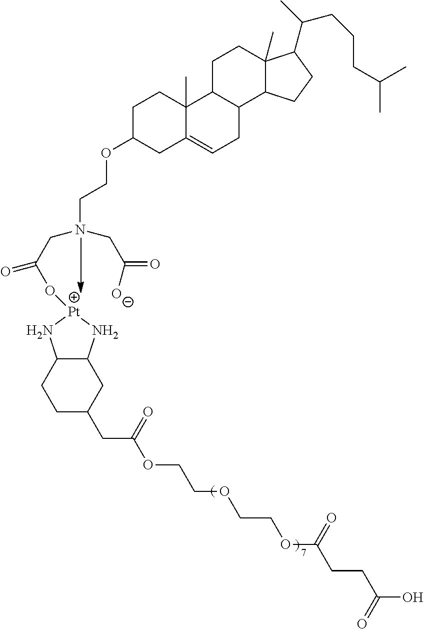
or a pharmaceutically acceptable salt thereof.

48. A method of inducing an immune response using the compound of claim 47.
